(19) United States
(12) Patent Application Publication
(10) Pub. No.: US 2005/0108791 A1
(43) Pub. Date: May 19, 2005

(54) Transgenic plants with improved phenotypes

(76) Inventor: Michael D. Edgerton, St. Louis, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/732,923

(22) Filed: Dec. 10, 2003

Related U.S. Application Data
(63) Continuation-in-part of application No. 10/310,154, filed on Dec. 4, 2002.
(60) Provisional application No. 60/337,358, filed on Dec. 4, 2001.
(60) Provisional application No. 60/449,054, filed on Feb. 22, 2003.

(51) Int. Cl. A01H 1/00; C12N 15/82; A01H 5/00

(52) U.S. Cl. 800/284; 800/320.1

(57) ABSTRACT

The present invention is directed to seed from a transgenic plant, wherein the genome of said seed comprises an exogenous polynucleotide comprising a functional portion of an encoding region for a polypeptide provided herein, and wherein plants grown from said seed exhibit an enhanced phenotype as compared to the phenotype of a control plant. Of particular interest are plants wherein the enhanced phenotype is increased yield. Exogenous polynucleotides of the present invention include recombinant polynucleotides providing for expression of mRNA encoding a polypeptide, and recombinant polynucleotides providing for expression of mRNA complementary to at least a portion of an mRNA native to the target plant for use in gene suppression.

### CROSS-REFERENCE TO RELATED APPLICATIONS

This application is a continuation-in-part of prior application Ser. No. 10/310,154 filed Dec. 4, 2002, which application claims priority under 35 U.S.C. §119(e) of U.S. Provisional Application No. 60/337,358 filed Dec. 4, 2001, the disclosure of which application is incorporated herein by reference in its entirety.

### INCORPORATION OF SEQUENCE LISTING

Two copies of the sequence listing (Copy 1 and Copy 2) and a computer readable form (CRF) of the sequence listing, all on CD-ROMs, each containing the file named Pa_{—}00613.rpt, which is 84,936,704 bytes (measured in MS-WINDOWS) and was created on Nov. 3, 2003, are herein incorporated by reference.

### INCORPORATION OF TABLES

Two copies of Tables 1-3 on CD-ROMs, each containing the file named pa_{—}00613.txt, which is 3,008,512 bytes (measured in MS-WINDOWS) and was created on Nov. 3, 2003, are herein incorporated by reference.

### FIELD OF THE INVENTION

Disclosed herein are seeds from transgenic plants, wherein the genome of said seed comprises recombinant polynucleotides, the expression of which results in the production of transgenic plants with enhanced phenotypes.

### BACKGROUND OF THE INVENTION

Transgenic plants with improved agronomic traits such as yield, pest resistance, herbicide tolerance, improved seed compositions, and the like are desired by both farmers and consumers. Although considerable efforts in plant breeding have provided significant gains in desired phenotypes, the ability to introduce specific DNA into plant genomes provides further opportunities for generation of plants with improved and/or unique phenotypes. The ability to develop transgenic plants with improved traits depends in part on the identification of genes that are useful in recombinant DNA constructs for production of transformed plants with improved properties.

### SUMMARY OF THE INVENTION

The present invention is directed to seed from a transgenic plant line, wherein said seed comprises in its genome a recombinant polynucleotide providing for expression or suppression of a polypeptide provided herein. Of particular interest is seed from a transgenic plant line, wherein said seed may be grown to produce plants having increased yield as compared to the yield of a control plant. Increased yield may be characterized as plant yield increase under non-stress conditions, or by plant yield increase under one or more environmental stress conditions. The invention also provides transgenic seed for plant lines having other enhanced phenotypes, such as enhanced plant morphology, plant physiology or seed component phenotype as compared to a corresponding phenotype of a control plant line. Of particular interest in the present invention is seed from transgenic crop plants, preferably maize (corn—Zea mays) or soybean (soy—Glycine max) plants. Other plants of interest in the present invention for production of transgenic seed that can be grown to provide plants having enhanced properties include, without limitation, cotton, canola, wheat, sunflower, sorghum, alfalfa, barley, millet, rice, tobacco, fruit and vegetable crops, and turfgrass.

In one aspect, this invention relates to the generation of transgenic plants by transformation with recombinant polynucleotides, and the identification of transgenic plants comprising such recombinant polynucleotides and having enhanced phenotypes. Of particular interest are transgenic plants that exhibit an improvement in a plant trait that is a component of yield. This aspect of the invention employs recombinant polynucleotides for expression of polypeptides that are useful for imparting desired traits to the transformed plants and recombinant polynucleotides for expression of homologs of such polypeptides as described herein. Exemplary polynucleotides which encode polypeptides of interest in the present invention are provided as SEQ ID NO:1 through SEQ ID NO:339. Sequences of the polypeptides of interest are provided as SEQ ID NO:340 through SEQ ID NO:678, and sequences of exemplary homolog polypeptides are provided as SEQ ID NO:679 through SEQ ID NO:24149. Tables 1-3 identifying the sequences of the present invention and their homologs are provided on the CD-ROM filed herewith.

Also of interest are recombinant polynucleotides that provide for suppression of expression of a target gene in a transgenic plant host using gene suppression methods, such as antisense or RNAi. Any of the polynucleotides provided herein as SEQ ID NO:1 through SEQ ID NO:339 may be used in such recombinant polynucleotides for gene suppression. Of particular interest are recombinant polynucleotides for gene suppression in maize, wherein said polynucleotide targets gene suppression of the corn aquaportin RS81 protein SEQ ID NO:8 or the retinoblastoma-related protein 1 provided as SEQ ID NO:70.

Thus, the present invention also comprises recombinant polynucleotides. Recombinant polynucleotides exemplified herein comprise a promoter functional in a plant cell operably joined to a DNA segment comprising encoding sequence for a polypeptide provided herein, or a homolog thereof. Such molecules are useful for production of transgenic plants having at least one improved property as the result of expression of a polypeptide of this invention or suppression of expression of a polypeptide described herein.

Also considered in the present invention is a method of producing a plant having an improved property, wherein the method comprises transforming a plant with a recombinant polynucleotide providing for expression or suppression of a polypeptide provided herein, and growing said transformed plant. In one aspect, the recombinant polynucleotide comprises a promoter functional in a plant cell operably joined to a DNA segment comprising encoding sequence for a polypeptide provided herein. The polynucleotide may be oriented with respect to the promoter to provide for transcription of sense or antisense RNA, or a combination of sense and antisense RNA, such as for use in RNAi methods of gene suppression. Of particular interest are uses of such methods to generate transgenic crop plants having increased yield.

Another aspect of the invention provides fragments of the polynucleotides of the present invention for use, for example as probes or molecular markers. Such fragments comprise at least 15 consecutive nucleotides in a sequence selected from the group consisting of SEQ ID NO:1 through SEQ ID NO:339 and complements thereof. Polynucleotide fragments of the present invention are useful as primers for PCR amplification and in hybridization assays such as transcription profiling assays, marker assays, or crop identity assays, including, for example, high throughput assays where the oligonucleotides are present in high density on a substrate, such as for example in microarrays.

### DETAILED DESCRIPTION OF THE INVENTION

The present invention is directed to seed from a transgenic plant, wherein the genome of said seed comprises an exogenous polynucleotide comprising a functional portion of an encoding region for a polypeptide provided herein, and wherein plants grown from said seed exhibit an enhanced phenotype as compared to the phenotype of a control plant. Of particular interest are plants wherein the enhanced phenotype is increased yield. Exogenous polynucleotides of the present invention include recombinant polynucleotides providing for expression of mRNA encoding a polypeptide, and recombinant polynucleotides providing for expression of mRNA complementary to at least a portion of an mRNA native to the target plant for use in gene suppression.

As used herein, a “transgenic plant” is one whose genome has been altered by the incorporation of exogenous genetic material, e.g. by transformation as described herein. The term “transgenic plant” is used to refer to the plant produced from an original transformation event, or progeny from later generations or crosses of a plant so transformed, so long as the progeny contains the exogenous genetic material in its genome. By “exogenous” is meant that a nucleic acid molecule, for example, a recombinant polynucleotide, originates from outside the plant into which it is introduced. An exogenous nucleic acid molecule may comprise naturally or non-naturally occurring polynucleotides, and may be derived from any organism, including the same or a different plant species than that into which it is introduced.

“Recombinant polynucleotide” refers in the present invention to a polynucleotide having a genetically engineered modification introduced through manipulation via mutagenesis, restriction enzymes, and the like. Recombinant polynucleotides may comprise DNA segments obtained from different sources, or DNA segments obtained from the same source, but which have been manipulated to join DNA segments which do not naturally exist in the joined form. A recombinant polynucleotide may exist outside of the cell, for example as a PCR fragment, or integrated into a genome, such as a plant genome.

As used herein, a “functional portion” of an encoding region for a polypeptide provided herein is a sufficient portion of the encoding region to provide the desired activity. Where expression of protein is desired, a functional portion will generally comprise the entire coding region for the polypeptide, although certain deletions, truncations, rearrangements and the like of the polypeptide may also maintain, or in some cases improve, the desired activity. One skilled in the art is aware of methods to screen for such desired modifications and such polypeptides are considered within the scope of the present invention. Where gene suppression methods are employed, smaller portions of the encoding region may be used to produce the desired effect.

“Enhanced phenotype” as used herein refers to a measurable improvement in a crop trait including, but not limited to, yield increase, including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. Many agronomic traits can affect “yield”, including without limitation, plant height, pod number, pod position on the plant, number of intemodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill.

Also of interest is the generation of transgenic plants that demonstrate enhanced phenotypic properties that may or may not confer an increase in overall plant yield. Such properties include enhanced plant morphology, plant physiology or enhanced components of the mature seed harvested from the transgenic plant. Of particular interest are enhancements in seed oil, tocopherol, protein and starch components, including increases in the quantity of any of these components, alterations in the ratios of any of these components, or production of new types of these components that do not exist in the seed from control plants. By way of example, increases in total tocopherol content are desirable, as are increases in the relative percentage of a-tocopherol produced by plants.

A “control plant” as used in the present invention is a plant used to compare against a transgenic plant grown from transgenic seed provided herein, to identify an enhanced phenotype in said transgenic plant. A suitable control plant may be a non-transgenic plant of the parental line used to generate a transgenic plant herein. A control plant may in some cases be a transgenic plant line that comprises an empty vector or marker gene, but does not contain the recombinant polynucleotide of the present invention that is expressed in the transgenic plant being evaluated. In general, a control plant is a plant of the same line or variety as the transgenic plant being tested.

“Increased yield” of a transgenic plant of the present invention may be evidenced and measured in a number of ways, including test weight, seed number per plant, seed weight, seed number per unit area (i.e. seeds, or weight of seeds, per acre), bushels per acre, tonnes per acre, tons per acre, kilo per hectare. For example, maize yield may be measured as production of shelled corn kernels per unit of production area, e.g. in bushels per acre or metric tons per hectare, often reported on a moisture adjusted basis, e.g. at 15.5% moisture. Increased yield may result from improved utilization of key biochemical compounds, such as nitrogen, phosphorous and carbohydrate, or from improved responses to environmental stresses, such as cold, heat, drought, salt, and attack by pests or pathogens. Polynucleotides of the present invention may also be used to provide plants having improved growth and development, and ultimately increased yield, as the result of modified expression of plant growth regulators or modification of cell cycle or photosynthesis pathways.

“Expression” as used herein refers to transcription of DNA to produce RNA. The resulting RNA may be without limitation mRNA encoding a protein, antisense RNA that is complementary to an mRNA encoding a protein, or an RNA transcript comprising a combination of sense and antisense gene regions, such as for use in RNAi technology. Expression as used herein may also refer to production of encoded protein from mRNA.

“Gene suppression” is used herein to refer to reduction or suppression of expression of a target protein in a host cell as the result of transcription of a recombinant polynucleotide provided herein, wherein the polynucleotide is oriented with respect to a promoter to provide for production of RNA having a gene silencing effect, such as antisense RNA or interfering RNA (RNAi).

Transgenic Plants and Seed

Transgenic plant seed provided by this invention may be grown to generate transgenic plants having an enhanced phenotype as compared to an appropriate control line. Such seed is obtained by screening transformed plants for enhanced phenotypes resulting from the introduction of a recombinant polynucleotide into the genomic DNA of tissue from a parental line. The recombinant polynucleotide is introduced into the genome to produce transgenic cells that can be cultured into transgenic plants having an enhanced phenotype as compared to the parental line or other appropriate control. Such transgenic cells are cultured into transgenic plants that produce progeny transgenic seed. Preferably, multiple transgenic plants (events) comprising the recombinant polynucleotides are evaluated, e.g. from 2 to 20 or more transgenic events, to identify a desired enhanced phenotype. Although the design of a recombinant polynucleotide is based on a rational expectation of a phenotypic modification, the present invention also contemplates that unexpected, yet desired enhanced phenotypes may be obtained.

Transgenic plants grown from transgenic seed provided herein demonstrate improved phenotypes that contribute to increased yield or other increased plant value, including, for example, improved seed quality. Of particular interest are plants having altered cell division, enhanced plant growth and development, stress tolerance, including tolerance to abiotic and biotic stress, altered seed or flower development, improved light response, and enhanced carbon and/or nitrogen metabolism, transport or utilization properties.

Yield enhancements by modification of cell division may be obtained, for example, by expression of cyclins, cytokinins, cyclin activating kinases or E2F or suppression of retinoblastoma 1.

Plant growth and development enhancements may be obtained, for example, by modification of expression of F box proteins or heterotrimeric G proteins, by modification of steroid biosynthesis and signaling or plant architecture, and by modification of activity of key plant development components, such as elongation factors, growth regulators and various transcription factors.

Stress tolerance enhancements may be obtained, for example by modification of expression of genes involved in heat tolerance, such as HSP90 and HSF genes; genes involved in cold tolerance, such as cold induced genes including SEQ ID NO:147 and SEQ ID NO:168 through SEQ ID NO:176, and fatty acid desaturase genes; genes associated with improved water use efficiency, such as Arabidopsis transcription factor G975 and crop homologs of G975; genes involved in disease resistance, including yeast superkiller (SKI) genes and plant superkiller homologs, or pest tolerance; genes associated with oxidative stress tolerance, such as provided as SEQ ID NO:241 through SEQ ID NO:272; genes associated with phospholipid signaling, jasmonate biosynthesis or flavonoid biosynthesis, or genes encoding phosphoinositide binding proteins, such as SEQ ID NO:331 through SEQ ID NO:335.

Seed development enhancements may be obtained, for example by modification of nitrate transport, modification of nucellin like proteins related to dsc1 and modification of expression of SET domain proteins, such as for alteration of endosperm or embryo size, or production of apomixis.

Light response enhancements may be obtained, for example by modification of expression of phytochrome or genes involved in phytochrome regulation or signal transduction genes such as provided as SEQ ID NO:23 through SEQ ID NO:31, SEQ ID NO:53 through SEQ ID NO:56, SEQ ID NO:59, SEQ ID NO:98, SEQ ID NO:11 through SEQ ID NO:113, SEQ ID NO:207, SEQ ID NO:215, SEQ ID NO:216, SEQ ID NO:230, SEQ ID NO:240, SEQ ID NO:277 and SEQ ID NO:311 through SEQ ID NO:315.

Flower development enhancements may be obtained, for example by modification of expression of genes related to flowering time such as provided herein as SEQ ID NO:40 through SEQ ID NO:43 and SEQ ID NO:326 through SEQ ID NO:328 and corn ear development, such as provided herein as SEQ ID NO:17 and SEQ ID NO:213.

Nitrogen utilization enhancements, including improved seed or grain quality, may be obtained, for example by modification of expression of genes involved in nitrogen assimilation, metabolism or transport.

Plant enhancements by alteration of source and/or sink properties are also considered in the present invention and may be obtained, for example, by improvements to sucrose production and/or transport, such as by expression of SEQ ID NO:279 through SEQ ID NO:283 and SEQ ID NO:298 through SEQ ID NO:308, or by modification of carbon partitioning.

Also of interest are plants having increased yield as the result of expression of genes, that are transcriptionally regulated in a manner that correlates with high yield, or by expression of homologs of such genes.

Polypeptides useful for generation of transgenic plants having enhanced properties are described in Table 4 below and provided herein as SEQ ID NO:340 through SEQ ID NO:678. Column headings in Table 4 refer to the following information:

“PEP SEQ ID NO” refers to a particular amino acid sequence in the Sequence Listing

“PHE ID” refers to an arbitrary number used to identify a particular recombinant polynucleotide corresponding to the translated protein encoded by the polynucleotide.

“NUC SEQ ID NO” refers to a particular nucleic acid sequence in the Sequence Listing which defines a polynucleotide used in a recombinant polynucleotide of this invention.

“GENE NAME” refers to a common name for the recombinant polynucleotide.

“GENE EFFECT” refers to the effect of the expressed polypeptide in providing yield improvement or other enhanced property

“CODING SEQUENCE” referS to peptide coding segments of the polynucleotide.

“SPECIES” refers to the organism from which the polynucleotide DNA was derived.

TABLE 4

| PEP | | NUC | | | | |
|---|---|---|---|---|---|---|
| SEQ | | SEQ | | | | |
| ID | | ID | | | | |
| NO | Phe ID | NO | Gene Name | Gene Effect | CODING SEQUENCE | Species |
| 340 | PHE0000001 | 1 | maize cellulose | Cold tolerance | 113-3061 | Zea mays |
| | | | synthase (eskimo 2) | | | |
| 341 | PHE0000006 | 2 | Arabidopsis RAV2/G9 | Root mass | 81-1136 | Arabidopsis |
| | | | | | | thaliana |
| 342 | PHE0000007 | 3 | rice G9-like 1 | Root mass | 336-1430 | Oryza sativa |
| 343 | PHE0000008 | 4 | rice G9-like 2 | Root mass | 572-1522 | Oryza sativa |
| 344 | PHE0000010 | 5 | rice G975 | Water use | 201-283, 516-1161 | Oryza sativa |
| | | | | efficiency | | |
| 345 | PHE0000278 | 6 | corn G975 | Water use | 41-679 | Zea mays |
| | | | | efficiency | | |
| 346 | PHE0000011 | 7 | corn Glossy15 | Water use | 385-1722 | Zea mays |
| | | | | efficiency | | |
| 347 | PHE0000012 | 8 | corn aquaporin RS81 | Root mass | 1-747 | Zea mays |
| 348 | PHE0000014 | 9 | rice cycD2 | Cell division | 13-324, 623-709, 813-911, | Oryza sativa |
| | | | | | 1003-1204, 1314-1438, | |
| | | | | | 1529-1774 | |
| 349 | PHE0000215 | 10 | invW | Sucrose | 1108-1489, 1813-2684, | Oryza sativa |
| | | | | production/transport | 6105-6266, 6417-6658, | |
| 350 | PHE0000015 | 11 | rice GCR1 | Cell division | 312-500, 1123-1154, 1384-1553, | Oryza sativa |
| | | | | | 2048-2163, 2724-2825, | |
| | | | | | 2946-3002, 3331-3474, | |
| | | | | | 3930-4000, 4118-4223 | |
| 351 | PHE0000016 | 12 | corn Knotted1 | Cell division | 181-1257 | Zea mays |
| 352 | PHE0000018 | 13 | corn AAA-ATPase 2 | Plastid division | 104-2533 | Zea mays |
| 353 | PHE0000019 | 14 | rice AOX1b | Cold tolerance | 4531-4851, 5011-5139, | Oryza sativa |
| | | | (alternative oxidase) | | 6072-6560, 6663-6722 | |
| 354 | PHE0000020 | 15 | Emericella nidulans | Cold tolerance | 2189-2442, 2492-2783, | Emericella |
| | | | alxA | | 2843-3352 | nidulans |
| 355 | PHE0000022 | 16 | corn AAP6-like | Nitrogen transport | 96-1547 | Zea mays |
| 356 | PHE0000024 | 17 | corn unknown protein | Flower | 441-2390 | Zea mays |
| | | | | development | | |
| 357 | PHE0000025 | 18 | corn GRF1-like | Plant growth and | 55-1470 | Zea mays |
| | | | protein | development | | |
| 358 | PHE0000026 | 19 | rice GRF1 | Plant growth and | 193-1380 | Oryza sativa |
| | | | | development | | |
| 359 | PHE0000227 | 20 | soy omega-3 fatty acid | Cold tolerance | 138-1496 | Glycine max |
| | | | desaturase | | | |
| 360 | PHE0000258 | 21 | AtFAD7 | Cold tolerance | 132-1472 | Arabidopsis |
| | | | | | | thaliana |
| 361 | PHE0000259 | 22 | AtFAD8 | Cold tolerance | 61-1368 | Arabidopsis |
| | | | | | | thaliana |
| 362 | PHE0000049 | 23 | rice phyA with corn | Light response | 4626-6690, 6913-7729, | Oryza sativa |
| | | | phyC intron 1 | | 8011-8307, 8410-8617 | |
| 363 | PHE0000027 | 24 | sorghum phyA with | Light response | 238-3633 | Sorghum |
| | | | corn phyC intron 1 | | | bicolor |
| 364 | PHE0000028 | 25 | rice phyB with corn | Light response | 67-3582 | Oryza sativa |
| | | | phyC intron 1 | | | |
| 365 | PHE0000029 | 26 | sorghum phyB with | Light response | 429-2640, 3333-4140, | Sorghum |
| | | | corn phyC intron 1 | | 5819-6112, 7491-7713 | bicolor |
| 366 | PHE0000030 | 27 | rice phyC with corn | Light response | 1036-3100, 3205-4021, | Oryza sativa |
| | | | phyC intron 1 | | 4418-4711, 5272-5509 | |
| 367 | PHE0000031 | 28 | sorghum phyC with | Light response | 303-3710 | Sorghum |
| | | | corn phyC intron 1 | | | bicolor |
| 368 | PHE0000032 | 29 | rice PF1 | Light response | 35-676 | Oryza sativa |
| 369 | PHE0000033 | 30 | rice GT2 | Light response | 58-2271 | Oryza sativa |
| 370 | PHE0000034 | 31 | Synechocystis | Light response | 9-992 | Synechocystis |
| | | | biliverdin reductase | | | sp. PCC 6803 |
| 371 | PHE0000038 | 32 | corn cycD2.1 | Cell division | 125-1156 | Zea mays |
| 372 | PHE0000039 | 33 | corn nph1 | Light response | 415-3150 | Zea mays |
| 373 | PHE0000040 | 34 | corn hemoglobin 1 | Stress tolerance | 172-669 | Zea mays |
| 374 | PHE0000043 | 35 | rice cyclin 2 | Cell division | 148-1407 | Oryza sativa |
| 375 | PHE0000044 | 36 | rice cycC | Cell division | 97-870 | Oryza sativa |
| 376 | PHE0000045 | 37 | rice cycB2 | Cell division | 74-1336 | Oryza sativa |
| 377 | PHE0000046 | 38 | rice cycA1 | Cell division | 97-1623 | Oryza sativa |
| 378 | PHE0000047 | 39 | rice cycB5 | Cell division | 292-361, 1019-1347, 1447-1572, | Oryza sativa |
| | | | | | 1657-1908, 2059-2217, | |
| | | | | | 2315-2493, 3276-3432 | |
| 379 | PHE0000244 | 40 | corn SVP-like | Flower | 177-860 | Zea mays |
| | | | | development | | |
| 380 | PHE0000245 | 41 | corn SVP-like | Flower | 93-791 | Zea mays |
| | | | | development | | |
| 381 | PHE0000246 | 42 | soy SVP-like | Flower | 96-713 | Glycine max |
| | | | | development | | |
| 382 | PHE0000247 | 43 | soy jointless-like | Flower | 60-674 | Glycine max |
| | | | | development | | |
| 383 | PHE0000106 | 44 | corn cycA1 | Cell division | 107-1633 | Zea mays |
| 384 | PHE0000050 | 45 | corn cycA2 | Cell division | 107-1222 | Zea mays |
| 385 | PHE0000051 | 46 | corn cycB2 | Cell division | 137-1408 | Zea mays |
| 386 | PHE0000052 | 47 | corn cycB5 | Cell division | 82-1518 | Zea mays |
| 387 | PHE0000382 | 48 | LIB3279-180-C9_FLI - | Cell division | 114-1385 | Zea mays |
| | | | maize cyclin III | | | |
| 388 | PHE0000053 | 49 | corn cycB4 | Cell division | 254-1579 | Zea mays |
| 389 | PHE0000054 | 50 | corn cycD3.2 | Cell division | 220-1380 | Zea mays |
| 390 | PHE0000055 | 51 | corn cycDx.1 | Cell division | 218-1180 | Zea mays |
| 391 | PHE0000056 | 52 | corn cycDl.1 | Cell division | 288-1334 | Zea mays |
| 392 | PHE0000057 | 53 | corn mt NDK - | Light response | 60-725 | Zea mays |
| | | | LIB189022Q1E1E9 | (Phytochrome?) | | |
| 393 | PHE0000058 | 54 | corn cp NDK - | Light response | 103-816 | Zea mays |
| | | | 700479629 | | | |
| 394 | PHE0000059 | 55 | corn NDK - | Light response | 49-495 | Zea mays |
| | | | LIB3597020Q1K6C3 | | | |
| 395 | PHE0000060 | 56 | corn NDK - | Light response | 162-608 | Zea mays |
| | | | 700241377 | | | |
| 396 | PHE0000062 | 57 | sRAD54 - with NLS | Homologous | 437-3556 | Synechocystis |
| | | | | recombination | | sp. PCC 6803 |
| 397 | PHE0000063 | 58 | T4 endonuclease VII | Homologous | 603-1148 | coliphage T4 |
| | | | (gp49) - with NLS | recombination | | |
| 398 | PHE0000064 | 59 | corn NDPK - fC- | Light response | 91-624 | Zea mays |
| | | | zmemLIB3957015Q1K6H6 | | | |
| 399 | PHE0000065 | 60 | TOR1 | Nitrogen | 302-7714 | Saccharomyces |
| | | | | assimilation | | cerevisiae |
| 400 | PHE0000292 | 61 | corn eIF-5A | Plant growth and | 85-564 | Zea mays |
| | | | | development | | |
| 401 | PHE0000067 | 62 | yeast eIF-5A | Plant growth and | 569-1042 | Saccharomyces |
| | | | | development | | cerevisiae |
| 402 | PHE0000068 | 63 | yeast deoxyhypusine | Plant growth and | 173-1336 | Saccharomyces |
| | | | synthase | development | | cerevisiae |
| 403 | PHE0000069 | 64 | yeast L5 | Plant growth and | 987-1880 | Saccharomyces |
| | | | | development | | cerevisiae |
| 404 | PHE0000070 | 65 | yeast ornithine | Plant growth and | 576-1976 | Saccharomyces |
| | | | decarboxylase | development | | cerevisiae |
| 405 | PHE0000071 | 66 | rice exportin 4-like | Plant growth and | 501-750, 1257-1417, 1735-1800, | Oryza sativa |
| | | | | development | 3104-3218, 3318-3427, | |
| | | | | | 3525-3620, 7587-7744, | |
| | | | | | 7828-7915, 8565-8669, | |
| | | | | | 8774-8878, 9421-9450, | |
| | | | | | 9544-9656, 9732-9819, | |
| | | | | | 9961-10180, 11034-11164, | |
| | | | | | 12058-12204, | |
| | | | | | 12770-12898, | |
| | | | | | 12975-13073, | |
| | | | | | 13221-13259, | |
| | | | | | 14674-14823 | |
| 406 | PHE0000072 | 67 | yeast S- | Plant growth and | 415-1605 | Saccharomyces |
| | | | adenosylmethionine | development | | cerevisiae |
| | | | decarboxylase | | | |
| 407 | PHE0000073 | 68 | corn S- | Plant growth and | 268-1365 | Zea mays |
| | | | adenosylmethionine | development | | |
| | | | decarboxylase 1 | | | |
| 408 | PHE0000074 | 69 | corn S- | Plant growth and | 581-1780 | Zea mays |
| | | | adenosylmethionine | development | | |
| | | | decarboxylase 2 | | | |
| 409 | PHE0000075 | 70 | retinoblastoma-related | Cell division | 37-2634 | Zea mays |
| | | | protein 1 | | | |
| 410 | PHE0000076 | 71 | C1 protein | Cell division | 49-843 | Wheat dwarf |
| | | | | | | virus |
| 411 | PHE0000077 | 72 | yeast flavohemoglobin - | Nitric oxide | 1695-2894 | Saccharomyces |
| | | | mitochondrial | signaling | | cerevisiae |
| 412 | PHE0000009 | 73 | Arabidopsis G975 | Water use | 58-654 | Arabidopsis |
| | | | | efficiency | | thaliana |
| 413 | PHE0000079 | 74 | CUT1 | Water use | 372-1082, 1176-1946 | Oryza sativa |
| | | | | efficiency | | |
| 414 | PHE0000082 | 75 | corn cycB3 | Cell division | 88-1425 | Zea mays |
| 415 | PHE0000083 | 76 | PDR5 | Disease resistance | 1552-6087 | Saccharomyces |
| | | | | (cercosporin | | cerevisiae |
| | | | | tolerance) | | |
| 416 | PHE0000084 | 77 | rice cyclin H | Cell division | 235-1227 | Oryza sativa |
| 417 | PHE0000085 | 78 | rice cdc2+/CDC28− | Cell division | 173-1447 | Oryza sativa |
| | | | related protein kinase | | | |
| 418 | PHE0000086 | 79 | Cdk-activating kinase 1 | Cell division | 14-1240 | Glycine max |
| 419 | PHE0000089 | 80 | CHL1 | Nitrogen | 85-1857 | Arabidopsis |
| | | | | uptake/Seed | | thaliana |
| | | | | development | | |
| 420 | PHE0000090 | 81 | NTR1 | Nitrogen | 144-1898 | Oryza sativa |
| | | | | uptake/Seed | | |
| | | | | development | | |
| 421 | PHE0000091 | 82 | Zm SET domain 2 | Seed development | 101-1009 | Zea mays |
| 422 | PHE0000092 | 83 | Zm SET domain 1 | Seed development | 528-1544 | Zea mays |
| 423 | PHE0000095 | 84 | HSF1 | Heat | 1017-3518 | Saccharomyces |
| | | | | tolerance/Water | | cerevisiae |
| | | | | use efficiency | | |
| 424 | PHE0000096 | 85 | Zm HSP101 | Heat | 436-1773, 1878-2159, | Zea mays |
| | | | | tolerance/Water | 2281-2621, 2711-2990, | |
| | | | | use efficiency | 3079-3276, 3371-3670 | |
| 425 | PHE0000098 | 86 | E. coli clpB | Heat | 557-3130 | Escherichia coli |
| | | | | tolerance/Water | | |
| | | | | use efficiency | | |
| 426 | PHE0000099 | 87 | Synechocystis clpB | Heat | 316-2931 | Synechocystis |
| | | | | tolerance/Water | | sp. PCC 6803 |
| | | | | use efficiency | | |
| 427 | PHE0000100 | 88 | Xylella clpB | Heat | 187-2769 | Xylella |
| | | | | tolerance/Water | | fastidiosa |
| | | | | use efficiency | | |
| 428 | PHE0000101 | 89 | corn cycD3.1 | Cell division | 250-1422 | Zea mays |
| 429 | PHE0000102 | 90 | AnFPPS (farnesyl- | Glyphosphate | 146-1186 | Emericella |
| | | | pyrophosphate | tolerance | | nidulans |
| | | | synthetase) | | | |
| 430 | PHE0000103 | 91 | OsFPPS | Glyphosphate | 42-1103 | Oryza sativa |
| | | | | tolerance | | |
| 431 | PHE0000104 | 92 | 700331819_FLI - corn | Glyphosphate | 313-1377 | Zea mays |
| | | | FPPS 2 | tolerance | | |
| 432 | PHE0000105 | 93 | corn cycD1.2 | Cell division | 229-1275 | Zea mays |
| 433 | PHE0000107 | 94 | corn cycD1.3 | Cell division | 206-1252 | Zea mays |
| 434 | PHE0000108 | 95 | ASH1 | Stress tolerance | 61-801 | Arabidopsis |
| | | | | | | thaliana |
| 435 | PHE0000109 | 96 | rice ASH1-like1 | Stress tolerance | 136-1008 | Oryza sativa |
| 436 | PHE0000110 | 97 | rice MtN2-like | Stress tolerance | 425-464, 546-582, 672-783, | Oryza sativa |
| | | | | | 812-898, 988-1149, | |
| | | | | | 1556-1675, 1776-1952 | |
| 437 | PHE0000111 | 98 | PAS domain kinase | Light response | 358-2613 | Zea mays |
| 438 | PHE0000114 | 99 | Su(var) 3-9-like | Seed development | 71-814 | Zea mays |
| 439 | PHE0000115 | 100 | Receiver domain | Cell division | 277-1002 | Zea mays |
| | | | (RR3-like) 7 | | | |
| 440 | PHE0000116 | 101 | Receiver domain | Cell division | 188-2245 | Zea mays |
| | | | (ARR2-like) 1 | | | |
| 441 | PHE0000117 | 102 | Receiver domain | Cell division | 112-2238 | Zea mays |
| | | | (TOC1-like) 2 | | | |
| 442 | PHE0000118 | 103 | Receiver domain | Cell division | 84-1976 | Zea mays |
| | | | (TOC1-like) 3 | | | |
| 443 | PHE0000119 | 104 | Receiver domain | Cell division | 39-1931 | Zea mays |
| | | | (ARR2-like) 4 | | | |
| 444 | PHE0000120 | 105 | Receiver domain | Cell division | 61-1812 | Zea mays |
| | | | (RR11-like) 5 | | | |
| 445 | PHE0000121 | 106 | Receiver domain | Cell division | 391-1116 | Zea mays |
| | | | (RR3-like) 6 | | | |
| 446 | PHE0000122 | 107 | Receiver domain | Cell division | 335-1066 | Zea mays |
| | | | (RR3-like) 8 | | | |
| 447 | PHE0000123 | 108 | Receiver domain 9 | Cell division | 55-759 | Zea mays |
| 448 | PHE0000124 | 109 | ZmRR2 | Cell division | 154-624 | Zea mays |
| 449 | PHE0000125 | 110 | Receiver domain | Cell division | 374-722, 791-2019 | Zea mays |
| | | | (TOC1-like) 10 | | | |
| 450 | PHE0000126 | 111 | corn HY5-like | Light response | 32-541 | Zea mays |
| 451 | PHE0000127 | 112 | scarecrow 1 (PAT1- | Light response | 295-1929 | Zea mays |
| | | | like) | | | |
| 452 | PHE0000128 | 113 | scarecrow 2 | Light response | 153-1934 | Zea mays |
| 453 | PHE0000133 | 114 | G protein b subunit | Plant growth and | 90-1229 | Zea mays |
| | | | | development/Stress | | |
| | | | | tolerance | | |
| 454 | PHE0000152 | 115 | 14-3-3-like protein 2 | Nitrogen | 85-861 | Glycine max |
| | | | | assimilation | | |
| 455 | PHE0000153 | 116 | 14-3-3-like protein D | Nitrogen | 42-824 | Glycine max |
| | | | | assimilation | | |
| 456 | PHE0000154 | 117 | 14-3-3 protein 1 | Nitrogen | 49-834 | Glycine max |
| | | | | assimilation | | |
| 457 | PHE0000155 | 118 | Rice FAP1-like | Nitrogen | 654-1862, 2310-2426, | Oryza sativa |
| | | | protein | assimilation | 3407-3492, 3590-3752, | |
| | | | | | 3845-3890, 4476-4522, | |
| | | | | | 4985-5191, 5306-5392, | |
| | | | | | 5473-5640 | |
| 458 | PHE0000156 | 119 | rice TAP42-like | Nitrogen | 199-1338 | Oryza sativa |
| | | | | assimilation | | |
| 459 | PHE0000158 | 120 | BMH1 | Nitrogen | 79-882 | Saccharomyces |
| | | | | assimilation | | cerevisiae |
| 460 | PHE0000159 | 121 | rice chloroplastic | Yield associated | 41-1261 | Oryza sativa |
| | | | fructose-1,6- | genes | | |
| | | | bisphosphatase | | | |
| 461 | PHE0000160 | 122 | E. coli fructose-1,6- | Yield associated | 208-1206 | Escherichia coli |
| | | | bisphosphatase | genes | | |
| 462 | PHE0000161 | 123 | Synechocystis | Yield associated | 1-1164 | Synechocystis |
| | | | fructose-1,6- | genes | | sp. PCC 6803 |
| | | | bisphosphatase F-I | | | |
| 463 | PHE0000162 | 124 | Synechocystis | Yield associated | 480-1523 | Synechocystis |
| | | | fructose-1,6- | genes | | sp. PCC 6803 |
| | | | bisphosphatase F-II | | | |
| 464 | PHE0000164 | 125 | Yeast RPT5 | Yield associated | 883-2187 | Saccharomyces |
| | | | | genes | | cerevisiae |
| 465 | PHE0000165 | 126 | Yeast RRP5 | Yield associated | 331-5520 | Saccharomyces |
| | | | | genes | | cerevisiae |
| 466 | PHE0000166 | 127 | Rice CBP-like gene | Yield associated | 277-436, 479-1524, 1790-2065, | Oryza sativa |
| | | | | genes | 2150-2425, 3134-3262, | |
| | | | | | 3380-3580, 3683-3825, | |
| | | | | | 3905-4190, 4294-4433, | |
| | | | | | 4711-4789, 4874-4929, | |
| | | | | | 5754-5946 | |
| 467 | PHE0000167 | 128 | rice BAB09754 | Yield associated | 616-903, 1848-1940, 2046-2165, | Oryza sativa |
| | | | | genes | 2254-2355, 2443-2693, | |
| | | | | | 2849-2994, 3165-3363, | |
| | | | | | 3475-4141, 4438-4770, | |
| | | | | | 5028-5309 | |
| 468 | PHE0000168 | 129 | LIB3061-001-H7_FLI | Yield associated | 309-1037 | Zea mays |
| | | | | genes | | |
| 469 | PHE0000169 | 130 | maize p23 | Heat | 106-708 | Zea mays |
| | | | | tolerance/Water | | |
| | | | | use efficiency | | |
| 470 | PHE0000170 | 131 | maize cyclophilin | Heat | 99-1757 | Zea mays |
| | | | | tolerance/Water | | |
| | | | | use efficiency | | |
| 471 | PHE0000172 | 132 | yeast SIT1 | Heat | 361-2130 | Saccharomyces |
| | | | | tolerance/Water | | cerevisiae |
| | | | | use efficiency | | |
| 472 | PHE0000173 | 133 | yeast CNS1 | Heat | 762-1919 | Saccharomyces |
| | | | | tolerance/Water | | cerevisiae |
| | | | | use efficiency | | |
| 473 | PHE0000176 | 134 | RNAse S | Phosphate uptake | 85-771 | Zea mays |
| 474 | PHE0000177 | 135 | maize ecto-apyrase | Phosphate uptake | 210-2312 | Zea mays |
| 475 | PHE0000178 | 136 | PHO5 | Phosphate uptake | 1-1404 | Saccharomyces |
| | | | | | | cerevisiae |
| 476 | PHE0000179 | 137 | high affinity phosphate | Phosphate uptake | 105-1703 | Glycine max |
| | | | translocator | | | |
| 477 | PHE0000180 | 138 | high affinity phosphate | Phosphate uptake | 128-1750 | Zea mays |
| | | | translocator | | | |
| 478 | PHE0000181 | 139 | Xylella citrate | Phosphate uptake | 256-1545 | Xylella |
| | | | synthase | | | fastidiosa |
| 479 | PHE0000182 | 140 | E. coli citrate synthase | Phosphate uptake | 309-1592 | Escherichia coli |
| 480 | PHE0000183 | 141 | rice citrate synthase | Phosphate uptake | 105-1523 | Oryza sativa |
| 481 | PHE0000184 | 142 | citrate synthase | Phosphate uptake | 56-1564 | Zea mays |
| 482 | PHE0000185 | 143 | citrate synthase | Phosphate uptake | 153-1691 | Glycine max |
| 483 | PHE0000186 | 144 | maize ferritin 2 | Stress tolerance | 3-758 | Zea mays |
| 484 | PHE0000187 | 145 | maize ferritin 1 | Stress tolerance | 34-795 | Zea mays |
| 485 | PHE0000188 | 146 | E. coli cytoplasmic | Stress tolerance | 245-742 | Escherichia coli |
| | | | ferritin | | | |
| 486 | PHE0000190 | 147 | corn LEA3 | Cold tolerance | 171-755 | Zea mays |
| 487 | PHE0000192 | 148 | soy HSF | Heat | 23-1114 | Glycine max |
| | | | | tolerance/Water | | |
| | | | | use efficiency | | |
| 488 | PHE0000193 | 149 | soy HSF | Heat | 93-992 | Glycine max |
| | | | | tolerance/Water | | |
| | | | | use efficiency | | |
| 489 | PHE0000204 | 150 | deoxyhypusine | Plant growth and | 26-1129 | Glycine max |
| | | | synthase | development | | |
| 490 | PHE0000219 | 151 | thylakoid carbonic | Photosynthesis | 62-994 | Chlamydomonas |
| | | | anhydrase, cah3 | | | reinhardtii |
| 491 | PHE0000216 | 152 | thylakoid carbonic | Photosynthesis | 49-843 | Nostoc |
| | | | anhydrase, ecaA | | | PCC7120 |
| 492 | PHE0000217 | 153 | Chlamydomonas | Photosynthesis | 156-1232 | Chlamydomonas |
| | | | reinhardtii envelope | | | reinhardtii |
| | | | protein LIP-36G1 | | | |
| 493 | PHE0000218 | 154 | psbO transit | Photosynthesis | 271-1674 | Synechococcus |
| | | | peptide::Synechococcus | | | sp. PCC 7942 |
| | | | sp. PCC 7942 ictB | | | |
| 494 | PHE0000220 | 155 | corn RNase PH | Disease resistance | 86-805 | Zea mays |
| 495 | PHE0000221 | 156 | SKI2 | Disease resistance | 1351-5211 | Saccharomyces |
| | | | | | | cerevisiae |
| 496 | PHE0000222 | 157 | SKI3 | Disease resistance | 793-5091 | Saccharomyces |
| | | | | | | cerevisiae |
| 497 | PHE0000223 | 158 | SKI4 | Disease resistance | 323-1201 | Saccharomyces |
| | | | | | | cerevisiae |
| 498 | PHE0000224 | 159 | SKI6 | Disease resistance | 1007-1747 | Saccharomyces |
| | | | | | | cerevisiae |
| 499 | PHE0000225 | 160 | SKI7 | Disease resistance | 279-2519 | Saccharomyces |
| | | | | | | cerevisiae |
| 500 | PHE0000226 | 161 | rice SKI7-like | Disease resistance | 464-884, 1132-1287, 2103-2252, | Oryza sativa |
| | | | | | 2353-2487, 2957-3288, | |
| | | | | | 3399-3509, 3596-4095, | |
| | | | | | 4350-4518, 4783-5022, | |
| | | | | | 5097-5228, 5315-5449 | |
| 501 | PHE0000228 | 162 | Synechocystis cobA wcp | Nitrogen | 70-801 | Synechocystis |
| | | | transit peptide | metabolism | | sp. PCC 6803 |
| 502 | PHE0000229 | 163 | Xylella tetrapyrrole | Nitrogen | 1-774 | Xylella |
| | | | methylase with transit | metabolism | | fastidiosa |
| | | | peptide | | | |
| 503 | PHE0000230 | 164 | maize | Nitrogen | 15-1286 | Zea mays |
| | | | uroporphyrinogen III | metabolism | | |
| | | | methyltransferase | | | |
| 504 | PHE0000231 | 165 | nucellin-like protein | Seed development | 122-1594 | Zea mays |
| 505 | PHE0000232 | 166 | nucellin-like protein | Seed development | 76-1605 | Zea mays |
| 506 | PHE0000233 | 167 | nucellin-like protein | Seed development | 195-1628 | Zea mays |
| 507 | PHE0000234 | 168 | soy LEA protein | Cold tolerance | 6-704 | Glycine max |
| 508 | PHE0000235 | 169 | dehydrin-like protein | Cold tolerance | 33-710 | Glycine max |
| 509 | PHE0000237 | 170 | dehydrin 3 | Cold tolerance | 84-584 | Zea mays |
| 510 | PHE0000238 | 171 | probable lipase | Cold tolerance | 98-967 | Zea mays |
| 511 | PHE0000239 | 172 | yeast GRE1 | Cold tolerance | 1024-1527 | Saccharomyces |
| | | | | | | cerevisiae |
| 512 | PHE0000240 | 173 | yeast STF2 | Cold tolerance | 683-934 | Saccharomyces |
| | | | | | | cerevisiae |
| 513 | PHE0000241 | 174 | yeast SIP18 | Cold tolerance | 376-855 | Saccharomyces |
| | | | | | | cerevisiae |
| 514 | PHE0000242 | 175 | yeast YBM6 | Cold tolerance | 744-1130 | Saccharomyces |
| | | | | | | cerevisiae |
| 515 | PHE0000243 | 176 | yeast HSP12 | Cold tolerance | 282-611 | Saccharomyces |
| | | | | | | cerevisiae |
| 516 | PHE0000249 | 177 | corn allene oxide | Stress | 111-1556 | Zea mays |
| | | | synthase | tolerance/Disease | | |
| | | | | resistance | | |
| 517 | PHE0000250 | 178 | corn COI1-like | Stress | 139-1911 | Zea mays |
| | | | | tolerance/Disease | | |
| | | | | resistance | | |
| 518 | PHE0000251 | 179 | corn TIR1-like | Plant growth and | 113-1906 | Zea mays |
| | | | | development | | |
| 519 | PHE0000252 | 180 | corn COI1-like | Stress | 130-1923 | Zea mays |
| | | | | tolerance/Disease | | |
| | | | | resistance | | |
| 520 | PHE0000253 | 181 | COI1-like | Stress | 389-2368 | Zea mays |
| | | | | tolerance/Disease | | |
| | | | | resistance | | |
| 521 | PHE0000254 | 182 | F-box protein | Plant growth and | 123-1304 | Glycine max |
| | | | | development | | |
| 522 | PHE0000255 | 183 | F-box protein | Plant growth and | 228-1916 | Glycine max |
| | | | | development | | |
| 523 | PHE0000256 | 184 | corn 1- | Stress | 61-1011 | Zea mays |
| | | | aminocyclopropane-1- | tolerance/Disease | | |
| | | | carboxylate oxidase | resistance | | |
| 524 | PHE0000257 | 185 | rice 1- | Stress | 2-1465 | Oryza sativa |
| | | | aminocyclopropane-1 | tolerance/Disease | | |
| | | | carboxylate synthase | resistance | | |
| 525 | PHE0000260 | 186 | S52650 - | Cold tolerance | 643-1719 | Synechocystis |
| | | | Synechocystis desB | | | sp. PCC 6803 |
| 526 | PHE0000261 | 187 | yeast glutamate | Stress tolerance | 33-1790 | Saccharomyces |
| | | | decarboxylase | | | cerevisiae |
| 527 | PHE0000262 | 188 | cytochrome P450-like | Plant growth and | 29-1495 | Zea mays |
| | | | protein | development | | |
| 528 | PHE0000263 | 189 | cytochrome P450 | Plant growth and | 141-1637 | Zea mays |
| | | | | development | | |
| 529 | PHE0000264 | 190 | cytochrome P450-like | Plant growth and | 104-1657 | Zea mays |
| | | | | development | | |
| 530 | PHE0000265 | 191 | CYP90 protein | Plant growth and | 81-1589 | Zea mays |
| | | | | development | | |
| 531 | PHE0000266 | 192 | cytochrome P450 | Plant growth and | 92-1648 | Zea mays |
| | | | DWARF3 | development | | |
| 532 | PHE0000267 | 193 | cytochrome P450 | Plant growth and | 134-1543 | Zea mays |
| | | | | development | | |
| 533 | PHE0000268 | 194 | rice receptor protein | Plant growth and | 183-476, 706-735, 2796-6734 | Oryza sativa |
| | | | kinase | development | | |
| 534 | PHE0000269 | 195 | soy E2F-like | Cell division | 80-1117 | Glycine max |
| 535 | PHE0000270 | 196 | nuclear matrix | Cell division | 243-3371 | Zea mays |
| | | | constituent protein | | | |
| 536 | PHE0000271 | 197 | OsE2F1 | Cell division | 93-1403 | Oryza sativa |
| 537 | PHE0000272 | 198 | corn GCR1 | Cell division | 74-1036 | Zea mays |
| 538 | PHE0000273 | 199 | soy mlo-like | Plant growth and | 15-1532 | Glycine max |
| | | | | development/Stress | | |
| | | | | tolerance | | |
| 539 | PHE0000274 | 200 | soy mlo-like | Plant growth and | 48-1841 | Glycine max |
| | | | | development/Stress | | |
| | | | | tolerance | | |
| 540 | PHE0000275 | 201 | rice G alpha 1 | Plant growth and | 106-1248 | Oryza sativa |
| | | | | development/Stress | | |
| | | | | tolerance | | |
| 541 | PHE0000276 | 202 | soy G-gamma subunit | Plant growth and | 210-536 | Glycine max |
| | | | | development/Stress | | |
| | | | | tolerance | | |
| 542 | PHE0000277 | 203 | wheat G28-like | Disease resistance | 65-877 | Triticum |
| | | | | | | aestivum |
| 543 | PHE0000279 | 204 | sorghum proline | Nitrogen transport | 16-1341 | Sorghum |
| | | | permease | | | bicolor |
| 544 | PHE0000280 | 205 | rice AA transporter | Nitrogen transport | 61-1485 | Oryza sativa |
| 545 | PHE0000282 | 206 | SET-domain protein- | Seed development | 478-3045 | Zea mays |
| | | | like | | | |
| 546 | PHE0000283 | 207 | scarecrow 6 | Light response | 520-2145 | Zea mays |
| 547 | PHE0000284 | 208 | menage a trois-like | Cell division | 164-745 | Zea mays |
| 548 | PHE0000286 | 209 | oryzacystatin | Pest tolerance | 108-527 | Oryza sativa |
| 549 | PHE0000287 | 210 | Similar to cysteine | Pest tolerance | 18-767 | Oryza sativa |
| | | | proteinase inhibitor | | | |
| 550 | PHE0000288 | 211 | cysteine proteinase | Pest tolerance | 135-461 | Sorghum |
| | | | inhibitor | | | bicolor |
| 551 | PHE0000289 | 212 | Zm-GRF1 (GA | Plant growth and | 96-1202 | Zea mays |
| | | | responsive factor) | development | | |
| 552 | PHE0000290 | 213 | ZmSE001-like | Flower | 253-2115 | Zea mays |
| | | | | development | | |
| 553 | PHE0000291 | 214 | deoxyhypusine | Plant growth and | 54-1163 | Zea mays |
| | | | synthase | development | | |
| 554 | PHE0000293 | 215 | gibberellin response | Light response | 131-2020 | Zea mays |
| | | | modulator | | | |
| 555 | PHE0000294 | 216 | scarecrow-like protein | Light response | 266-1948 | Zea mays |
| 556 | PHE0000295 | 217 | ubiquitin-conjugating | Yield associated | 114-599 | Zea mays |
| | | | enzyme-like protein | genes | | |
| 557 | PHE0000296 | 218 | unknown protein | Yield associated | 90-785 | Zea mays |
| | | | recognized by | genes | | |
| | | | PF01169 | | | |
| 558 | PHE0000297 | 219 | 26S protease | Yield associated | 57-1343 | Oryza sativa |
| | | | regulatory subunit 6A | genes | | |
| | | | homolog | | | |
| 559 | PHE0000298 | 220 | rice p23 co-chaperone | Heat | 68-706 | Oryza sativa |
| | | | | tolerance/Water | | |
| | | | | use efficiency | | |
| 560 | PHE0000299 | 221 | corn p23 co-chaperone | Heat | 71-565 | Zea mays |
| | | | | tolerance/Water | | |
| | | | | use efficiency | | |
| 561 | PHE0000300 | 222 | rice p23 co-chaperone | Heat | 124-642 | Oryza sativa |
| | | | | tolerance/Water | | |
| | | | | use efficiency | | |
| 562 | PHE0000301 | 223 | corn p23 co-chaperone | Heat | 90-617 | Zea mays |
| | | | | tolerance/Water | | |
| | | | | use efficiency | | |
| 563 | PHE0000302 | 224 | putative purple acid | Phosphate uptake | 22-1038 | Oryza sativa |
| | | | phosphatase precursor | | | |
| 564 | PHE0000303 | 225 | acid phosphatase type 5 | Phosphate uptake | 143-1186 | Zea mays |
| 565 | PHE0000304 | 226 | aleurone ribonuclease | Phosphate uptake | 47-814 | Oryza sativa |
| 566 | PHE0000305 | 227 | putative ribonuclease | Phosphate uptake | 55-888 | Zea mays |
| 567 | PHE0000306 | 228 | S-like RNase | Phosphate uptake | 15-770 | Zea mays |
| 568 | PHE0000307 | 229 | ribonuclease | Phosphate uptake | 95-781 | Zea mays |
| 569 | PHE0000308 | 230 | helix-loop-helix | Light response | 202-756 | Zea mays |
| | | | protein (PIF3-like) | | | |
| 570 | PHE0000309 | 231 | SKI4-like protein | Disease resistance | 36-632 | Zea mays |
| 571 | PHE0000310 | 232 | putative 3 | Disease resistance | 238-1098 | Zea mays |
| | | | exoribonuclease | | | |
| 572 | PHE0000311 | 233 | GF14-c protein | Nitrogen | 81-848 | Oryza sativa |
| | | | | assimilation | | |
| 573 | PHE0000312 | 234 | 14-3-3-like protein | Nitrogen | 6-785 | Oryza sativa |
| | | | | assimilation | | |
| 574 | PHE0000313 | 235 | rice eIF-(iso)4F | Nitrogen | 96-713 | Oryza sativa |
| | | | | assimilation | | |
| 575 | PHE0000314 | 236 | rice eIF-4F | Nitrogen | 46-726 | Oryza sativa |
| | | | | assimilation | | |
| 576 | PHE0000315 | 237 | sorghum eIF-(iso)4F | Nitrogen | 78-707 | Sorghum |
| | | | | assimilation | | bicolor |
| 577 | PHE0000316 | 238 | sorghum eIF-4F | Nitrogen | 9-668 | Sorghum |
| | | | | assimilation | | bicolor |
| 578 | PHE0000317 | 239 | rice FIP37-like | Nitrogen | 73-1128 | Oryza sativa |
| | | | | assimilation | | |
| 579 | PHE0000318 | 240 | scarecrow 17 | Light response | 441-2102 | Zea mays |
| 580 | PHE0000322 | 241 | maize catalase-1 | Stress tolerance | 208-1683 | Zea mays |
| 581 | PHE0000323 | 242 | maize catalase-3 | Stress tolerance | 30-1511 | Zea mays |
| 582 | PHE0000324 | 243 | ascorbate peroxidase | Stress tolerance | 197-1063 | Zea mays |
| 583 | PHE0000325 | 244 | corn GDI | Stress tolerance | 57-1397 | Zea mays |
| 584 | PHE0000326 | 245 | soy GDI | Stress tolerance | 45-1418 | Glycine max |
| 585 | PHE0000327 | 246 | corn rho GDI | Stress tolerance | 463-1203 | Zea mays |
| 586 | PHE0000328 | 247 | basic blue copper | Stress tolerance | 13-408 | Zea mays |
| | | | protein | | | |
| 587 | PHE0000329 | 248 | plantacyanin | Stress tolerance | 109-489 | Zea mays |
| 588 | PHE0000330 | 249 | basic blue copper | Stress tolerance | 83-463 | Glycine max |
| | | | protein | | | |
| 589 | PHE0000331 | 250 | Similar to blue copper | Stress tolerance | 323-868 | Zea mays |
| | | | protein precursor | | | |
| 590 | PHE0000332 | 251 | lamin | Stress tolerance | 62-646 | Zea mays |
| 591 | PHE0000333 | 252 | fC-zmfl700551169a- | Stress tolerance | 56-1105 | Zea mays |
| | | | allyl alcohol | | | |
| | | | dehydrogenase | | | |
| 592 | PHE0000334 | 253 | allyl alcohol | Stress tolerance | 103-1128 | Glycine max |
| | | | dehydrogenase | | | |
| 593 | PHE0000335 | 254 | allyl alcohol | Stress tolerance | 6-1079 | Zea mays |
| | | | dehydrogenase | | | |
| 594 | PHE0000336 | 255 | quinone | Stress tolerance | 47-1051 | Zea mays |
| | | | oxidoreductase | | | |
| 595 | PHE0000337 | 256 | E. nidulans cysA - | Stress tolerance | 384-1961 | Emericella |
| | | | AF029885 | | | nidulans |
| 596 | PHE0000338 | 257 | BAA18167 - | Stress tolerance | 801-1547 | Synechocystis |
| | | | Synechocystis cysE | | | sp. PCC 6803 |
| 597 | PHE0000339 | 258 | Synechocystis thiol- | Stress tolerance | 36-638 | Synechocystis |
| | | | specific antioxidant | | | sp. PCC 6803 |
| | | | protein - BAA10136 | | | |
| 598 | PHE0000340 | 259 | yeast TSA2 - | Stress tolerance | 108-698 | Saccharomyces |
| | | | NP_010741 | | | cerevisiae |
| 599 | PHE0000341 | 260 | yeast mTPx - Z35825 | Stress tolerance | 730-1512 | Saccharomyces |
| | | | | | | cerevisiae |
| 600 | PHE0000343 | 261 | yeast TPx III - | Stress tolerance | 657-1187 | Saccharomyces |
| | | | NP_013210 | | | cerevisiae |
| 601 | PHE0000345 | 262 | soy putative 2-cys | Stress tolerance | 160-939 | Glycine max |
| | | | peroxiredoxin | | | |
| 602 | PHE0000346 | 263 | soy peroxiredoxin | Stress tolerance | 104-745 | Glycine max |
| 603 | PHE0000347 | 264 | heat shock protein 26, | Stress tolerance | 117-836 | Zea mays |
| | | | plastid-localized | | | |
| 604 | PHE0000349 | 265 | heat shock protein | Stress tolerance | 112-735 | Zea mays |
| 605 | PHE0000350 | 266 | low molecular weight | Stress tolerance | 28-690 | Zea mays |
| | | | heat shock protein | | | |
| 606 | PHE0000351 | 267 | 18 kDa heat shock | Stress tolerance | 103-597 | Zea mays |
| | | | protein | | | |
| 607 | PHE0000352 | 268 | heat shock protein | Stress tolerance | 229-690 | Zea mays |
| | | | 16.9 | | | |
| 608 | PHE0000353 | 269 | HSP21-like protein | Stress tolerance | 73-696 | Zea mays |
| 609 | PHE0000354 | 270 | Opt1p - NP_012323 | Stress tolerance | 508-2904 | Saccharomyces |
| | | | | | | cerevisiae |
| 610 | PHE0000355 | 271 | SVCT2-like permease | Stress tolerance | 220-1779 | Zea mays |
| 611 | PHE0000356 | 272 | SVCT2-like permease | Stress tolerance | 34-1632 | Zea mays |
| 612 | PHE0000357 | 273 | maize tubby-like | Plant growth and | 519-1958 | Zea mays |
| | | | | development | | |
| 613 | PHE0000358 | 274 | maize tubby-like | Plant growth and | 517-1269 | Zea mays |
| | | | | development | | |
| 614 | PHE0000359 | 275 | soy HMG CoA | Stress tolerance | 80-1441 | Glycine max |
| | | | synthase | | | |
| 615 | PHE0000360 | 276 | yeast HMGS - X96617 | Stress tolerance | 220-1695 | Saccharomyces |
| | | | | | | cerevisiae |
| 616 | PHE0000361 | 277 | PAT1-like scarecrow 9 | Light response | 191-1900 | Zea mays |
| 617 | PHE0000362 | 278 | CDC28-related protein | Cell division | 198-1484 | Zea mays |
| | | | kinase | | | |
| 618 | PHE0000385 | 279 | H+ transporting | Metabolite | 176-2836 | Zea mays |
| | | | ATPase | transport | | |
| 619 | PHE0000386 | 280 | cation-transporting | Metabolite | 222-2168 | Zea mays |
| | | | ATPase | transport | | |
| 620 | PHE0000387 | 281 | yeast DRS2 (ALA1- | Metabolite | 170-4237 | Saccharomyces |
| | | | like) - L01795 | transport | | cerevisiae |
| 621 | PHE0000388 | 282 | S. pombe ALA1-like- | Metabolite | 56-3832 | Schizosaccharo |
| | | | CAA21897 | transport | | myces pombe |
| 622 | PHE0000389 | 283 | rice ALA1-like 1 - | Metabolite | 47-1538, 1619-1925, 3116-3824, | Oryza sativa |
| | | | BAA89544 | transport | 3920-4043, 4143-4362, | |
| | | | | | 4590-5048, 5937-6153 | |
| 623 | PHE0000390 | 284 | rice chloroplastic | Photosynthesis/Carbon | 136-1311 | Oryza sativa |
| | | | sedoheptulose-1,7- | partitioning | | |
| | | | bisphosphatase- | | | |
| 624 | PHE0000391 | 285 | rice cytosolic fructose- | Photosynthesis/Carbon | 171-1187 | Oryza sativa |
| | | | 1,6-bisphosphatase | partitioning | | |
| 625 | PHE0000392 | 286 | Wheat sedoheptulose- | Photosynthesis/Carbon | 14-1192 | Triticum |
| | | | 1,7-bisphosphatase | partitioning | | aestivum |
| 626 | PHE0000394 | 287 | sedoheptulose-1,7- | Photosynthesis/Carbon | 90-1238 | Chlorella |
| | | | bisphosphatase | partitioning | | sorokiniana |
| 627 | PHE0000395 | 288 | soy phantastica | Plant growth and | 275-1345 | Glycine max |
| | | | | development | | |
| 628 | PHE0000396 | 289 | soy phantastica 2 | Plant growth and | 178-1260 | Glycine max |
| | | | | development | | |
| 629 | PHE0000397 | 290 | maize rough sheath 1 | Plant growth and | 92-1144 | Zea mays |
| | | | | development | | |
| 630 | PHE0000398 | 291 | soy lg3-like 1 | Plant growth and | 103-1026 | Glycine max |
| | | | | development | | |
| 631 | PHE0000399 | 292 | soy rough sheath1-like 1 | Plant growth and | 144-1076 | Glycine max |
| | | | | development | | |
| 632 | PHE0000400 | 293 | soy G559-like | Plant growth and | 301-1560 | Glycine max |
| | | | | development | | |
| 633 | PHE0000401 | 294 | soy G1635-like 1 | Plant growth and | 28-888 | Glycine max |
| | | | | development | | |
| 634 | PHE0000402 | 295 | rice amino acid | Nitrogen transport | 89-1426 | Oryza sativa |
| | | | transporter-like protein | | | |
| 635 | PHE0000403 | 296 | corn amino acid | Nitrogen transport | 116-1453 | Zea mays |
| | | | permease | | | |
| 636 | PHE0000404 | 297 | rice proline transport | Nitrogen transport | 313-1731 | Oryza sativa |
| | | | protein | | | |
| 637 | PHE0000412 | 298 | corn monosaccharide | Sucrose transport | 75-1643 | Zea mays |
| | | | transporter 1 | | | |
| 638 | PHE0000413 | 299 | soy monosaccharide | Sucrose transport | 132-1685 | Glycine max |
| | | | transporter 3 | | | |
| 639 | PHE0000414 | 300 | corn monosaccharide | Sucrose transport | 141-1670 | Zea mays |
| | | | transporter 3 | | | |
| 640 | PHE0000415 | 301 | soy monosaccharide | Sucrose transport | 160-1899 | Glycine max |
| | | | transporter 1 | | | |
| 641 | PHE0000416 | 302 | corn monosaccharide | Sucrose transport | 74-1690 | Zea mays |
| | | | transporter 6 | | | |
| 642 | PHE0000418 | 303 | corn monosaccharide | Sucrose transport | 146-1744 | Zea mays |
| | | | transporter 4 | | | |
| 643 | PHE0000419 | 304 | soy monosaccharide | Sucrose transport | 63-1505 | Glycine max |
| | | | transporter 2 | | | |
| 644 | PHE0000420 | 305 | soy sucrose transporter | Sucrose transport | 63-1595 | Glycine max |
| 645 | PHE0000421 | 306 | corn sucrose | Sucrose transport | 76-1599 | Zea mays |
| | | | transporter 2 | | | |
| 646 | PHE0000422 | 307 | corn monosaccharide | Sucrose transport | 201-1763 | Zea mays |
| | | | transporter 8 | | | |
| 647 | PHE0000423 | 308 | corn monosaccharide | Sucrose transport | 93-1634 | Zea mays |
| | | | transporter 7 | | | |
| 648 | PHE0000425 | 309 | soy isoflavone | Stress tolerance | 45-1607 | Glycine max |
| | | | synthase | | | |
| 649 | PHE0000426 | 310 | soy ttg1-like 2 | Stress tolerance | 52-1059 | Glycine max |
| 650 | PHE0000427 | 311 | GATE5 - corn SPA1- | Light response | 227-3139 | Zea mays |
| | | | like 1 | | | |
| 651 | PHE0000428 | 312 | corn PIF3-like | Light response | 173-856 | Zea mays |
| 652 | PHE0000429 | 313 | soy Athb-2-like 1 | Light response | 78-932 | Glycine max |
| 653 | PHE0000430 | 314 | corn SUB1-like 1 | Light response | 44-1954 | Zea mays |
| 654 | PHE0000431 | 315 | soy GH3 protein | Light response | 42-1820 | Glycine max |
| 655 | PHE0000432 | 316 | corn 12- | Stress | 128-1240 | Zea mays |
| | | | oxophytodienoate | tolerance/Disease | | |
| | | | reductase 1 | resistance | | |
| 656 | PHE0000433 | 317 | corn 12-oxo- | Stress | 166-1242 | Zea mays |
| | | | phytodienoate | tolerance/Disease | | |
| | | | reductase-like 3 | resistance | | |
| 657 | PHE0000434 | 318 | corn 12- | Stress | 92-1210 | Zea mays |
| | | | oxophytodienoate | tolerance/Disease | | |
| | | | reductase-like 4 | resistance | | |
| 658 | PHE0000435 | 319 | corn hydroperoxide | Stress | 83-1594 | Zea mays |
| | | | lyase | tolerance/Disease | | |
| | | | | resistance | | |
| 659 | PHE0000436 | 320 | rice cns1-like | Heat | 121-1242 | Oryza sativa |
| | | | | tolerance/Water | | |
| | | | | use efficiency | | |
| 660 | PHE0000437 | 321 | corn HCH1-like 1 | Heat | 42-1100 | Zea mays |
| | | | | tolerance/Water | | |
| | | | | use efficiency | | |
| 661 | PHE0000438 | 322 | corn HOP-like 1 | Heat | 88-1830 | Zea mays |
| | | | | tolerance/Water | | |
| | | | | use efficiency | | |
| 662 | PHE0000439 | 323 | corn HOP-like 2 | Heat | 65-1261 | Zea mays |
| | | | | tolerance/Water | | |
| | | | | use efficiency | | |
| 663 | PHE0000440 | 324 | rice CHIP-like 1 | Heat | 121-939 | Oryza sativa |
| | | | | tolerance/Water | | |
| | | | | use efficiency | | |
| 664 | PHE0000441 | 325 | corn CHIP-like 2 | Heat | 115-939 | Zea mays |
| | | | | tolerance/Water | | |
| | | | | use efficiency | | |
| 665 | PHE0000451 | 326 | wheat SVP-like 1 | Flower | 149-736 | Triticum |
| | | | | development | | aestivum |
| 666 | PHE0000452 | 327 | corn SVP-like 3 | Flower | 75-749 | Zea mays |
| | | | | development | | |
| 667 | PHE0000453 | 328 | corn SVP-like 5 | Flower | 304-774, 956-1219 | Zea mays |
| | | | | development | | |
| 668 | PHE0000454 | 329 | fC-zmhuLIB3062- | Yield associated | 113-853 | Zea mays |
| | | | 044-Q1-K1-B8 | genes | | |
| 669 | PHE0000455 | 330 | corn E4/E8 binding | Yield associated | 253-2259 | Zea mays |
| | | | protein-like | genes | | |
| 670 | PHE0000469 | 331 | yeast YKL091c - | Stress tolerance | 110-1042 | Saccharomyces |
| | | | Z28091 | | | cerevisiae |
| 671 | PHE0000470 | 332 | corn Ssh1-like protein 1 | Stress tolerance | 57-1037 | Zea mays |
| 672 | PHE0000471 | 333 | corn Ssh1-like protein 3 | Stress tolerance | 89-841 | Zea mays |
| 673 | PHE0000472 | 334 | corn Ssh1-like protein 4 | Stress tolerance | 309-1196 | Zea mays |
| 674 | PHE0000473 | 335 | soy Ssh1-like protein 2 | Stress tolerance | 209-976 | Glycine max |
| | | | [ssh2] | | | |
| 675 | PHE0000484 | 336 | soy JMT-like protien 1 | Stress | 26-1135 | Glycine max |
| | | | | tolerance/Disease | | |
| | | | | resistance | | |
| 676 | PHE0000485 | 337 | corn JMT-like protein 1 | Stress | 39-1184 | Zea mays |
| | | | | tolerance/Disease | | |
| | | | | resistance | | |
| 677 | PHE0000486 | 338 | corn JMT-like protein 2 | Stress | 63-1208 | Zea mays |
| | | | | tolerance/Disease | | |
| | | | | resistance | | |
| 678 | PHE0000017 | 339 | corn AAA-ATPase 1 | Plastid division | 184-2214 | Zea mays |

Transgenic plants having enhanced phenotypes are identified from populations of plants transformed as described herein by evaluating the phenotype in a variety of assays to detect an enhanced phenotype. These assays also may take many forms, including but not limited to, analyses to detect changes in the chemical composition, morphology, biomass or physiological responses of the plant to stress conditions. Enhanced physiological properties in transgenic plants of the present invention may be identified by evaluation of responses to stress conditions, for example in assays using imposed stress conditions to detect improved responses to water stress, nitrogen deficiency, cold growing conditions, pathogen or insect attack or light deficiency, or alternatively, under naturally present stress conditions, for example under field conditions. Enhanced chemical compositions, such as nutritional composition of grain, may be detected by analysis, for example, of composition and content of seed protein, free amino acids, oil, free fatty acids, starch or tocopherols. Biomass measures may be made on greenhouse or field grown plants and may include such measurements as plant height, stem diameter, root and shoot dry weights, and, for corn plants, ear length and diameter

Phenotypic data on morphological changes may be collected by visual observation during the process of plant regeneration as well as in regenerated plants transferred to soil. Such phenotypic data includes characteristics such as normal plants, bushy plants, taller plants, thicker stalks, narrow leaves, striped leaves, knotted phenotype, chlorosis, albino, anthocyanin production, or altered tassels, ears or roots. Other enhanced phenotypes may be identified by measurements taken under field conditions, such as days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality.

To confirm hybrid yield in transgenic corn plants expressing genes of the present invention, it may be desirable to test hybrids over multiple years at multiple locations in a geographical location where maize is conventionally grown, e.g. in Iowa, Illinois or other locations in the midwestern United States, under “normal” field conditions as well as under stress conditions, e.g. under drought or population density stress.

Of particular interest in the present invention are corn and soybean plants. Other plants of interest in the present invention for production of transgenic seed that can be grown to provide plants having enhanced properties include, without limitation, cotton, canola, wheat, sunflower, sorghum, alfalfa, barley, millet, rice, tobacco, fruit and vegetable crops, and turfgrass.

Protein and Polypeptide Molecules

Polypeptides considered in the present invention are entire proteins or at least a sufficient portion of the entire protein to impart the relevant biological activity of the protein, e.g. enhanced plant phenotype. The term “protein” also includes molecules consisting of one or more polypeptide chains. Thus, a polypeptide useful in the present invention may constitute an entire protein having the desired biological activity, or may constitute a portion of an oligomeric protein having multiple polypeptide chains. Polypeptides useful for generation of transgenic plants having enhanced properties include the polypeptides provided herein as SEQ ID NO:340 through SEQ ID NO:678, as well as homologs of such polypeptides.

Homologs of the polypeptides of the present invention may be identified by comparison of the amino acid sequence of the polypeptide to amino acid sequences of polypeptides from the same or different plant sources, e.g. manually or by using known homology-based search algorithms such as those commonly known and referred to as BLAST, FASTA, and Smith-Waterman. As used herein, a homolog is a peptide from the same or a different organism that performs the same biological function as the polypeptide to which it is compared. An orthologous relation between two organisms is not necessarily manifest as a one-to-one correspondence between two genes, because a gene can be duplicated or deleted after organism phylogenetic separation, such as speciation. For a given polypeptide, there may be no ortholog or more than one ortholog. Other complicating factors include alternatively spliced transcripts from the same gene, limited gene identification, redundant copies of the same gene with different sequence lengths or corrected sequence. A local sequence alignment program, e.g. BLAST, can be used to search a database of sequences to find similar sequences, and the summary Expectation value (E-value) used to measure the sequence base similarity. As a polypeptide hit with the best E-value for a particular organism may not necessarily be an ortholog or the only ortholog, a reciprocal BLAST search is used in the present invention to filter hit sequences with significant E-values for ortholog identification. The reciprocal BLAST entails search of the significant hits against a database of polypeptide sequences from the base organism that are similar to the sequence of the query polypeptide. A hit is a likely ortholog, when the reciprocal BLAST's best hit is the query polypeptide itself or a polypeptide encoded by a duplicated gene after speciation. Thus, homolog is used herein to described polypeptides that are assumed to have functional similarity by inference from sequence base similarity. Homologs of the polypeptides of the present invention are described in Table 2 provided on the CD-ROM provided herewith, and disclosed as SEQ ID NO:679 through SEQ ID NO:24149.

A further aspect of the invention comprises functional homolog proteins which differ in one or more amino acids from those of a polypeptide provided herein as the result of one or more of the well-known conservative amino acid substitutions, e.g. valine is a conservative substitute for alanine and threonine is a conservative substitute for serine. Conservative substitutions for an amino acid within the native polypeptide sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the invention comprises polypeptides that differ in one or more amino acids from those of a described protein sequence as the result of deletion or insertion of one or more amino acids in a native sequence.

Homologs of the polypeptides provided herein will generally demonstrate significant identity with the polypeptides provided herein. Of particular interest are polypeptides having at least 50% sequence identity, more preferably at least about 70% sequence identity or higher, e.g. at least about 80% sequence identity with an amino acid sequence of SEQ ID NO:1 through SEQ ID NO:339. Of course useful polypeptides also include those with higher identity to such a polypeptide sequence, e.g. 90% to 99% identity. Identity of protein homologs is determnined by optimally aligning the amino acid sequence of a putative protein homolog with a defined amino acid sequence and by calculating the percentage of identical and conservatively substituted amino acids over the window of comparison. Preferentially, the window of comparison for determining identity is the entire polypeptide sequence disclosed herein, e.g. the full sequence of any of SEQ ID NO:340 through SEQ ID NO:678.

Recombinant Polynucleotides

The present invention contemplates the use of polynucleotides effective for imparting an enhanced phenotype to transgenic plants expressing said polynucleotides. Exemplary polynucleotides for use in the present invention are listed in Table 4 above and provided herein as SEQ ID NO:1 through SEQ ID NO:339. A subset of the nucleic molecules of this invention includes fragments of the disclosed polynucleotides consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group consisting of SEQ ID NO: 1 through SEQ ID NO:339, and find use, for example as probes and primers for detection of the polynucleotides of the present invention.

Also of interest in the present invention are variants of the polynucleotides provided herein. Such variants may be naturally occurring, including homologous polynucleotides from the same or a different species, or may be non-natural variants, for example polynucleotides synthesized using chemical synthesis methods, or generated using recombinant DNA techniques. Degeneracy of the genetic code provides the possibility to substitute at least one base of the protein encoding sequence of a gene with a different base without causing the amino acid sequence of the polypeptide produced from the gene to be changed. Hence, a polynucleotide useful in the present invention may have any base sequence that has been changed from SEQ ID NO:1 to SEQ ID NO:339 by substitution in accordance with degeneracy of the genetic code.

Homologs of the polynucleotides provided herein will generally demonstrate significant identity with the polynucleotides provided herein. A polynucleotide of the present invention is substantially identical to a reference polynucleotide if, when the sequences of the polynucleotides are optimally aligned there is about 60% nucleotide equivalence; more preferably 70%; more preferably 80% equivalence; more preferably 85% equivalence; more preferably 90%; more preferably 95%; and/or more preferably 98% or 99% equivalence over a comparison window. A comparison window is preferably at least 50-100 nucleotides, and more preferably is the entire length of the polynucleotide provided herein. Optimal alignment of sequences for aligning a comparison window may be conducted by algorithms; preferably by computerized implementations of these algorithms (for example, the Wisconsin Genetics Software Package Release 7.0-10.0, Genetics Computer Group, 575 Science Dr., Madison, Wis.). The reference polynucleotide may be a full-length molecule or a portion of a longer molecule. Preferentially, the window of comparison for determining polynucleotide identity of protein encoding sequences is the entire coding region.

In a preferred embodiment, a polynucleotide of the present invention is operatively linked in a recombinant polynucleotide to a promoter functional in a plant to provide for expression of the polynucleotide in the sense orientation such that a desired polypeptide is produced. Also considered are embodiments wherein a polynucleotide is operatively linked to a promoter functional in a plant to provide for expression of the polynucleotide in the antisense orientation such that a complementary copy of at least a portion of an mRNA native to the target plant host is produced. Such a transcript may contain both sense and antisense regions of a polynucleotide, for example where RNAi methods are used for gene suppression.

Recombinant polynucleotides of the present invention are assembled in recombinant DNA constructs using methods known to those of ordinary skill in the art. Thus, transgenic DNA constructs used for transforming plant cells will comprise a polynucleotide one desires to introduce into a target plant. Such constructs will also typically comprise a promoter operatively linked to said polynucleotide to provide for expression in the target plant. Other construct components may include additional regulatory elements, such as 5′ or 3′ untranslated regions (such as polyadenylation sites), intron regions, and transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens, caulimovirus promoters such as the cauliflower mosaic virus or figwort mosaic virus promoters. For instance, see U.S. Pat. Nos. 5,858,742 and 5,322,938 which disclose versions of the constitutive promoter derived from cauliflower mosaic virus (CaMV35S), U.S. Pat. No. 5,378,619 which discloses a Figwort Mosaic Virus (FMV) 35S promoter, U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, U.S. Pat. No. 6,175,060 which discloses phosphorus deficiency inducible promoters, U.S. patent application Publication 2002/0192813A1 which discloses 5′,3′ and intron elements useful in the design of effective plant expression vectors, U.S. patent application Ser. No. 09/078,972 which discloses a coixin promoter, U.S. patent application Ser. No. 09/757,089 which discloses a maize chloroplast aldolase promoter, all of which are incorporated herein by reference. These and numerous other promoters that function in plant cells are known to those skilled in the art and available for use in recombinant polynucleotides of the present invention to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers that can be used in accordance with the invention include elements from the CaMV 35S promoter, octopine synthase genes, the maize alcohol dehydrogenase gene, the maize shrunken 1 gene and promoters from non-plant eukaryotes.

In some aspects of the invention it is preferred that the promoter element in the DNA construct be capable of causing sufficient expression to result in the production of an effective amount of a polypeptide in water deficit conditions. Such promoters can be identified and isolated from the regulatory region of plant genes that are over expressed in water deficit conditions. Specific water-deficit-inducible promoters for use in this invention are derived from the 5′ regulatory region of genes identified as a heat shock protein 17.5 gene (HSP17.5), an HVA22 gene (HVA22), and a cinnamic acid 4-hydroxylase (CA4H) gene (CA4H) of Zea maize. Such water-deficit-inducible promoters are disclosed in U.S. provisional application Ser. No. 60/435,987, filed Dec. 20, 2002, incorporated herein by reference.

In other aspects of the invention, sufficient expression in plant seed tissues is desired to effect improvements in seed composition. Exemplary promoters for use for seed composition modification include promoters from seed genes such as napin (U.S. Pat. No. 5,420,034), oleosin, zein Z27 (Russell et al. (1997) Transgenic Res. 6(2):157-166), globulin 1 (Belanger et al (1991) Genetics 129:863-872), glutelin 1 (Russell (1997) supra), and peroxiredoxin antioxidant (Per1) (Stacy et al. (1996) Plant Mol Biol. 31(6): 1205-1216).

In still other aspects of the invention, preferential expression in plant green tissues is desired. Promoters of interest for such uses include those from genes such as SSU (Fischhoff et al. (1992) Plant Mol Biol. 20:81-93), aldolase and pyruvate orthophosphate dikinase (PPDK) (WO01/19976).

Recombinant constructs prepared in accordance with the invention will also generally include a 3′ untranlated DNA region that typically contains a polyadenylation sequence following the polynucleotide coding region. Examples of useful 3′ UTRs include those from the nopaline synthase gene of Agrobacterium tumefaciens (nos), a gene encoding the small subunit of a ribulose-1,5-bisphosphate carboxylase-oxygenase (rbcS), and the T7 transcript of Agrobacterium tumefaciens.

Constructs and vectors may also include a transit peptide for targeting of a gene target to a plant organelle, particularly to a chloroplast, leucoplast or other plastid organelle. For descriptions of the use of chloroplast transit peptides see U.S. Pat. No. 5,188,642 and U.S. Pat. No. 5,728,925, incorporated herein by reference. For description of the transit peptide region of an Arabidopsis EPSPS gene useful in the present invention, see Klee, H. J. et al (MGG (1987) 210:437-442).

The present invention also encompasses transgenic plants with stacked engineered traits, e.g. a crop having an enhanced phenotype resulting from expression of a recombinant polynucleotide provided herein, in combination with herbicide and/or pest resistance traits. For example, genes of the current invention can be stacked with other traits of agronomic interest, such as a trait providing herbicide resistance, for example a RoundUp Ready trait, or insect resistance, such as using a gene from Bacillus thuringensis to provide resistance against lepidopteran, coliopteran, homopteran, hemiopteran, and other insects. Herbicides for which resistance is useful in a plant include glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and gluphosinate herbicides. To illustrate the that production of transgenic plants with herbicide resistance is a capability of those of ordinary skill in the art reference is made to U.S. patent application publications 2003/0106096A1 and 2002/0112260A1 and U.S. Pat. Nos. 5,034,322; 5,776,760, 6,107,549 and 6,376,754, all of which are incorporated herein by reference. To illustrate that the production of transgenic plants with pest resistance is a capability of those of ordinary skill in the art reference is made to U.S. Pat. Nos. 5,250,515 and 5,880,275 which disclose plants expressing an endotoxin of Bacillus thuringiensis bacteria, to U.S. Pat. No. 6,506,599 which discloses control of invertebrates which feed on transgenic plants which express dsRNA for suppressing a target gene in the invertebrate, to U.S. Pat. No. 5,986,175 which discloses the control of viral pests by transgenic plants which express viral replicase, and to U.S. patent application Publication 2003/0150017 A1 which discloses control of pests by a transgenic plant which express a dsRNA targeted to suppressing a gene in the pest, all of which are incorporated herein by reference.

Plant Transformation Constructs and Methods

Constructs used for transforming plant cells will comprise the recombinant polynucleotide that one desires to introduce as well as various other elements as described above. It is also contemplated that one may employ multiple genes for expression of multiple polynucleotides for crop improvement provided herein or for expression of a polynucleotide provided herein and one or more other desirable genes on either the same or different vectors for transformation. In the latter case, the different vectors may be delivered concurrently to recipient cells if co-transformation into a single chromosomal location is desired. Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. Nos. 5,015,580; 5,550,318; 5,538,880; 5,914,451; 6,160,208; 6,399,861 and 6,403,865 and Agrobacterium-mediated transformation is described in U.S. Pat. Nos. 5,635,055; 5,824,877; 5,591,616; 5,981,840 and 6,384,301, all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is preferred to introduce heterologous DNA randomly, i.e. at a non-specific location, in the genome of a target plant line. In special cases it may be useful to target heterologous DNA insertion in order to achieve site-specific integration, e.g. to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695, both incorporated herein by reference.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. Nos. 6,194,636 and 6,232,526 and U.S. patent application Ser. No. 09/757,089, which are incorporated herein by reference.

In practice DNA is introduced into only a small percentage of target cells in any one experiment. Marker genes are used to provide an efficient system for identification of those cells that are stably transformed by receiving and integrating a transgenic DNA construct into their genomes. Preferred marker genes provide selective markers that confer resistance to a selective agent, such as an antibiotic or herbicide. Potentially transformed cells are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Useful selective marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known. It is also contemplated that combinations of screenable and selectable markers will be useful for identification of transformed cells. See PCT publication WO 99/61129 which discloses use of a gene fusion between a selectable marker gene and a screenable marker gene, e.g. an NPTII gene and a GFP gene.

Cells that survive exposure to the selective agent, or cells that have been scored positive in a screening assay, may be cultured in regeneration media and allowed to mature into plants. Developing plantlets can be transferred to soil less plant growth mix, and hardened off, e.g., in an environmentally controlled chamber at about 85% relative humidity, 600 ppm CO₂, and 25-250 microeinsteins m⁻² s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown to plants on solid media at about 19 to 28° C. After regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous recombinant polynucleotide. Useful assays include, for example, “molecular biological” assays, such as Southern and Northern blotting and PCR; “biochemical” assays, such as detecting the presence of RNA, e.g. double stranded RNA, or a protein product, e.g., by immunological means (ELISAs and Western blots) or by enzymatic function; plant part assays, such as leaf or root assays; and also, by analyzing the phenotype of the whole regenerated plant.

The present invention will be further illustrated by means of the following examples provided for illustration purposes only and in no way intended to limit the scope of the invention.

### EXAMPLES

### Example 1

### Constructs for Maize Transformation

A GATEWAY™ Destination (Invitrogen Life Technologies, Carlsbad, Calif.) plant expression vector, pMON65154, was constructed for use in preparation of constructs comprising recombinant polynucleotides for corn transformation. The elements of the expression vector are summarized in Table 5 below. Generally, pMON65154 comprises a selectable marker expression cassette comprising a Cauliflower Mosaic Virus 35S promoter operably linked to a gene encoding neomycin phosphotransferase II (nptII). The 3′ region of the selectable marker expression cassette comprises the 3′ region of the Agrobacterium tumefaciense nopaline synthase gene (nos) followed 3′ by the 3′ region of the potato proteinase inhibitor II (pinII) gene. The plasmid pMON 65154 further comprises a plant expression cassette into which a gene of interest may be inserted using GATEWAY™ cloning methods. The GATEWAY™ cloning cassette is flanked 5′ by a rice actin 1 promoter, exon and intron and flanked 3′ by the 3′ region of the potato pinII gene. Using GATEWAY™ methods, the cloning cassette may be replaced with a gene of interest. The vector pMON65154, and derivatives thereof comprising a gene of interest, are particularly useful in methods of plant transformation via direct DNA delivery, such as microprojectile bombardment.

TABLE 5
Elements of Plasmid pMON65154

| FUNCTION | ELEMENT | REFERENCE |
|---|---|---|
| Plant gene of interest | Rice actin 1 promoter | U.S. Pat. No. 5,641,876 |
| expression cassette | Rice actin 1 exon 1, intron 1 | U.S. Pat. No. 5,641,876 |
| | enhancer | |
| Gene of interest insertion | AttR1 | GATEWAY ™ |
| site | | Cloning Technology |
| | | Instruction Manual |
| | CmR gene | GATEWAY ™ |
| | | Cloning Technology |
| | | Instruction Manual |
| | ccdA, ccdB genes | GATEWAY ™ |
| | | Cloning Technology |
| | | Instruction Manual |
| | attR2 | GATEWAY ™ |
| | | Cloning Technology |
| | | Instruction Manual |
| Plant gene of interest | Potato pinII 3′ region | An et al. (1989) Plant Cell 1: |
| expression cassette | | 115-122 |
| Plant selectable marker | CaMV 35S promoter | U.S. Pat. No. 5,858,742 |
| expression cassette | nptII selectable marker | U.S. Pat. No. 5,858,742 |
| | nos 3′ region | U.S. Pat. No. 5,858,742 |
| | PinII 3′ region | An et al. (1989) Plant Cell 1: |
| | | 115-122 |
| Maintenance in E. coli | ColE1 origin of replication | |
| | F1 origin of replication | |
| | Bla ampicillin resistance | |

A similar plasmid vector, pMON72472, is constructed for use in Agrobacterium mediated methods of plant transformation. pMON72472 comprises the gene of interest plant expression cassette, GATEWAY™ cloning, and plant selectable marker expression cassettes present in pMON65154. In addition, left and right T-DNA border sequences from Agrobacterium are added to the plasmid (Zambryski et al. (1982)). The right border sequence is located 5′ to the rice actin 1 promoter and the left border sequence is located 3′ to the pinII 3′ sequence situated 3′ to the nptII gene. Furthermore, pMON72472 comprises a plasmid backbone to facilitate replication of the plasmid in both E. coli and Agrobacterium tumefaciens. The backbone has an oriV wide host range origin of DNA replication functional in Agrobacterium, a pBR322 origin of replication functional in E. coli, and a spectinomycin/stretptomycin resistance gene for selection in both E. coli and Agrobacterium.

Vectors similar to those described above may be constructed for use in Agrobacterium or microprojectile bombardment maize transformation systems where the rice actin 1 promoter in the plant expression cassette portion is replaced with other desirable promoters including, but not limited to a maize globulin 1 promoter, a maize oleosin promoter, a glutelin 1 promoter, an aldolase promoter, a zein Z27 promoter, a pyruvate orthophosphate dikinase (PPDK) promoter, a a soybean 7S alpha promoter, a peroxiredoxin antioxidant (Per1) promoter and a CaMV 35S promoter. Protein coding segments are amplified by PCR prior to insertion into vectors such as described above. Primers for PCR amplification can be designed at or near the start and stop codons of the coding sequence, in order to eliminate most of the 5′ and 3′ untranslated regions. For GATEWAY cloning methods, PCR products are tailed with attB1 and attB2 sequences, purified then recombined into a destination vectors to produce an expression vector for use in transformation.

Exemplary constructs for transformation of maize to produce plants having enhanced phenotypes are provided in Table 6 below. Column headings in Table 6 refer to the following information:

“SEQ ID NO” refers to a particular nucleic acid sequence in the Sequence Listing which defines a polynucleotide used in a recombinant polynucleotide of this invention.

“PHE ID” refers to an arbitrary number used to identify a particular recombinant polynucleotide corresponding to the translated protein encoded by the polynucleotide.

“NOM ID” refers to a particular construct comprising a polynucleotide of this invention.

“GENE NAME” refers to a common name for the recombinant polynucleotide.

“PROMOTER” provides the name of the promoter region driving expression of the polynucleotide

“TARGET” indicates if a chloroplast transit peptide is employed in the construct

“pMON” refers to an arbitrary number used to designate a particular recombinant DNA construct. Constructs are prophetic where no pMON is provided.

TABLE 6
Maize Transformation Constructs

| SEQ | | | | | | |
|---|---|---|---|---|---|---|
| ID | | | | | | |
| NO | Phe ID | Nom ID | Gene Name | Promoter | Target | pMON |
| 1 | PHE0000001 | 1 | maize cellulose synthase | rice actin | | — |
| 2 | PHE0000006 | 6 | Arabidopsis RAV2/G9 | rice actin | | PMON68861 |
| 3 | PHE0000007 | 7 | rice G9-like 1 | rice actin | | — |
| 4 | PHE0000008 | 8/4361 | rice G9-like 2 | rice actin | | PMON80526 |
| 5 | PHE0000010 | 13 | rice G975 | rice actin | | PMON67800 |
| 6 | PHE0000278 | 14 | corn G975 | rice actin | | PMON68886 |
| 7 | PHE0000011 | 17 | corn Glossy15 | rice actin | | — |
| 8 | PHE0000012 | 165 | corn aquaporin RS81 | rice actin | | PMON67808 |
| 8 | PHE0000012 | 166 | antisense corn aquaporin RS81 | rice actin | | PMON67806 |
| 9 | PHE0000014 | 22 | rice cycD2 | rice actin | | PMON80471 |
| 10 | PHE0000215 | 24 | invW | rice actin | | — |
| 11 | PHE0000015 | 25 | rice GCR1 | rice actin | | PMON80255 |
| 12 | PHE0000016 | 103 | corn Knotted1 | rice actin | | PMON67750 |
| 13 | PHE0000018 | 28 | corn AAA-ATPase 2 | rice actin | | — |
| 14 | PHE0000019 | 29 | rice AOX1b | rice actin | | PMON80879 |
| 15 | PHE0000020 | 30 | Emericella nidulans alxA | rice actin | | PMON81241 |
| 16 | PHE0000022 | 34 | corn AAP6-like | rice actin | | PMON67826 |
| 17 | PHE0000024 | 41 | corn unknown protein | rice actin | | PMON68354 |
| 18 | PHE0000025 | 44 | corn GRF1-like protein | rice actin | | PMON68396 |
| 19 | PHE0000026 | 45 | rice GRF1 | rice actin | | — |
| 20 | PHE0000227 | 46 | soy omega-3 fatty acid desaturase | rice actin | | PMON68376 |
| 21 | PHE0000258 | 47 | AtFAD7 | rice actin | | PMON68371 |
| 22 | PHE0000259 | 48 | AtFAD8 | rice actin | | PMON74404 |
| 23 | PHE0000049 | 52 | rice phyA with corn phyC intron 1 | rice actin | | PMON80912 |
| 24 | PHE0000027 | 53/4386 | sorghum phyA with corn phyC | rice actin | | PMON80920 |
| | | | intron 1 | | | |
| 25 | PHE0000028 | 54 | rice phyB with corn phyC intron 1 | rice actin | | — |
| 26 | PHE0000029 | 55 | sorghum phyB with corn phyC | rice actin | | — |
| | | | intron 1 | | | |
| 27 | PHE0000030 | 56 | rice phyC with corn phyC intron 1 | rice actin | | — |
| 28 | PHE0000031 | 57 | sorghum phyC with corn phyC | rice actin | | — |
| | | | intron 1 | | | |
| 29 | PHE0000032 | 58 | rice PF1 | rice actin | | PMON83627 |
| 30 | PHE0000033 | 4152 | rice GT2 | PPDK | | — |
| 31 | PHE0000034 | 60 | Synechocystis biliverdin | rice actin | | PMON67805 |
| | | | reductase | | | |
| 32 | PHE0000038 | 65 | corn cycD2.1 | rice actin | | PMON68383 |
| 33 | PHE0000039 | 67 | corn nph1 | rice actin | | PMON67807 |
| 34 | PHE0000040 | 71 | corn hemoglobin 1 | rice actin | | PMON67801 |
| 34 | PHE0000040 | 74 | corn hemoglobin 1 | rice actin | chloroplast | PMON77889 |
| 35 | PHE0000043 | 80 | rice cyclin 2 | rice actin | | PMON80322 |
| 36 | PHE0000044 | 81/4424 | rice cycC | rice actin | | PMON80482 |
| 37 | PHE0000045 | 82 | rice cycB2 | rice actin | | PMON81293 |
| 38 | PHE0000046 | 83/4425 | rice cycA1 | rice actin | | PMON78247 |
| 39 | PHE0000047 | 84 | rice cycB5 | rice actin | | — |
| 40 | PHE0000244 | 89 | corn SVP-like | rice actin | | PMON68372 |
| 41 | PHE0000245 | 90 | corn SVP-like | rice actin | | PMON68373 |
| 42 | PHE0000246 | 91 | soy SVP-like | rice actin | | PMON68374 |
| 43 | PHE0000247 | 92 | soy jointless-like | rice actin | | PMON68375 |
| 44 | PHE0000106 | 114/4427 | corn cycA1 | rice actin | | PMON69457 |
| 45 | PHE0000050 | 115 | corn cycA2 | rice actin | | — |
| 46 | PHE0000051 | 116 | corn cycB2 | rice actin | | PMON68859 |
| 47 | PHE0000052 | 117 | corn cycB5 | rice actin | | PMON67813 |
| 48 | PHE0000382 | 118 | LIB3279-180-C9_FLI - maize | rice actin | | PMON74401 |
| | | | cyclin III | | | |
| 49 | PHE0000053 | 119 | corn cycB4 | rice actin | | — |
| 50 | PHE0000054 | 120/4369 | corn cycD3.2 | rice actin | | PMON81815 |
| 51 | PHE0000055 | 121 | corn cycDx.1 | rice actin | | PMON68355 |
| 52 | PHE0000056 | 122 | corn cycD1.1 | rice actin | | PMON68364 |
| 53 | PHE0000057 | 124 | corn mt NDK - | rice actin | | PMON68350 |
| | | | LIB189022Q1E1E9 | | | |
| 54 | PHE0000058 | 125 | corn cp NDK - 700479629 | rice actin | | PMON68351 |
| 55 | PHE0000059 | 126 | corn NDK - | rice actin | | PMON68370 |
| | | | LIB3597020Q1K6C3 | | | |
| 56 | PHE0000060 | 127 | corn NDK - 700241377 | rice actin | | PMON68356 |
| 57 | PHE0000062 | 130 | sRAD54 - with NLS | rice actin | | — |
| 58 | PHE0000063 | 132 | T4 endonuclease VII (gp49) - | rice actin | | — |
| | | | with NLS | | | |
| 59 | PHE0000064 | 137 | corn NDPK - fC- | rice actin | | PMON67804 |
| | | | zmemLIB3957015Q1K6H6 | | | |
| 60 | PHE0000065 | 139/4405 | TOR1 | rice actin | | — |
| 61 | PHE0000292 | 142 | corn eIF-5A | rice actin | | PMON68888 |
| 62 | PHE0000067 | 143 | yeast eIF-5A | rice actin | | PMON67816 |
| 63 | PHE0000068 | 144 | yeast deoxyhypusine synthase | rice actin | | PMON67824 |
| 64 | PHE0000069 | 147 | yeast L5 | rice actin | | PMON67821 |
| 65 | PHE0000070 | 149 | yeast ornithine decarboxylase | rice actin | | PMON67825 |
| 66 | PHE0000071 | 151 | rice exportin 4-like | rice actin | | — |
| 67 | PHE0000072 | 152 | yeast S-adenosylmethionine | rice actin | | PMON67828 |
| | | | decarboxylase | | | |
| 68 | PHE0000073 | 153 | corn S-adenosylmethionine | rice actin | | PMON68357 |
| | | | decarboxylase 1 | | | |
| 69 | PHE0000074 | 154 | corn S-adenosylmethionine | rice actin | | PMON68352 |
| | | | decarboxylase 2 | | | |
| 70 | PHE0000075 | 155 | antisense retinoblastoma-related | rice actin | | — |
| | | | protein 1 | | | |
| 71 | PHE0000076 | 156 | C1 protein | rice actin | | PMON68851 |
| 72 | PHE0000077 | 157 | yeast flavohemoglobin - | rice actin | | PMON67827 |
| | | | mitochondrial | | | |
| 72 | PHE0000077 | 158 | yeast flavohemoglobin - | rice actin | chloroplast | PMON77890 |
| | | | mitochondrial | | | |
| 72 | PHE0000077 | 159 | yeast flavohemoglobin - | rice actin | | PMON75301 |
| | | | mitochondrial | | | |
| 73 | PHE0000009 | 164 | Arabidopsis G975 | rice actin | | PMON67803 |
| 74 | PHE0000079 | 169 | CUT1 | rice actin | | PMON67752 |
| 75 | PHE0000082 | 172 | corn cycB3 | rice actin | | — |
| 76 | PHE0000083 | 173 | PDR5 | rice actin | | PMON81229 |
| 77 | PHE0000084 | 174 | rice cyclin H | rice actin | | — |
| 78 | PHE0000085 | 175 | rice cdc2+/CDC28−related protein | rice actin | | PMON80475 |
| | | | kinase | | | |
| 79 | PHE0000086 | 176 | Cdk-activating kinase 1 | rice actin | | PMON67812 |
| 80 | PHE0000089 | 179/4360 | CHL1 | rice actin | | PMON80273 |
| 81 | PHE0000090 | 180/4387 | NTR1 | rice actin | | PMON80335 |
| 82 | PHE0000091 | 181 | Zm SET domain 2 | rice actin | | PMON68358 |
| 83 | PHE0000092 | 182 | Zm SET domain 1 | rice actin | | PMON68359 |
| 84 | PHE0000095 | 185 | HSF1 | rice actin | | PMON80915 |
| 85 | PHE0000096 | 186 | Zm HSP101 | rice actin | | — |
| 86 | PHE0000098 | 188 | E. coli clpB | rice actin | | PMON73168 |
| 87 | PHE0000099 | 189 | Synechocystis clpB | rice actin | | PMON80517 |
| 88 | PHE0000100 | 190 | Xylella clpB | rice actin | | PMON80917 |
| 89 | PHE0000101 | 191/4352 | corn cycD3.1 | rice actin | | PMON81811 |
| 90 | PHE0000102 | 192 | AnFPPS (farnesyl-pyrophosphate | rice actin | | PMON67815 |
| | | | synthetase) | | | |
| 91 | PHE0000103 | 193 | OsFPPS | rice actin | | PMON83631 |
| 92 | PHE0000104 | 194 | 700331819_FLI - corn FPPS 2 | rice actin | | PMON68608 |
| 93 | PHE0000105 | 195/4426 | corn cycD1.2 | rice actin | | PMON80329 |
| 94 | PHE0000107 | 197/4428 | corn cycD1.3 | rice actin | | PMON81259 |
| 95 | PHE0000108 | 198 | ASH1 | rice actin | | PMON67849 |
| 96 | PHE0000109 | 199 | rice ASH1-like1 | rice actin | | — |
| 97 | PHE0000110 | 200 | rice MtN2-like | rice actin | | PMON80473 |
| 98 | PHE0000111 | 201 | PAS domain kinase | rice actin | | — |
| 99 | PHE0000114 | 204 | Su(var) 3-9-like | rice actin | | PMON68361 |
| 100 | PHE0000115 | 205 | Receiver domain (RR3-like) 7 | rice actin | | PMON68362 |
| 101 | PHE0000116 | 206 | Receiver domain (ARR2-like) 1 | rice actin | | PMON68367 |
| 102 | PHE0000117 | 207 | Receiver domain (TOC1-like) 2 | rice actin | | PMON68368 |
| 103 | PHE0000118 | 208 | Receiver domain (TOC1-like) 3 | rice actin | | PMON67811 |
| 104 | PHE0000119 | 209 | Receiver domain (ARR2-like) 4 | rice actin | | PMON68363 |
| 105 | PHE0000120 | 210 | Receiver domain (RR11-like) 5 | rice actin | | PMON68853 |
| 106 | PHE0000121 | 211 | Receiver domain (RR3-like) 6 | rice actin | | PMON68854 |
| 107 | PHE0000122 | 212 | Receiver domain (RR3-like) 8 | rice actin | | PMON74402 |
| 108 | PHE0000123 | 213 | Receiver domain 9 | rice actin | | PMON68855 |
| 109 | PHE0000124 | 214 | ZmRR2 | rice actin | | PMON68856 |
| 110 | PHE0000125 | 215 | Receiver domain (TOC1-like) 10 | rice actin | | PMON68369 |
| 111 | PHE0000126 | 216 | corn HY5-like | rice actin | | PMON69458 |
| 112 | PHE0000127 | 217 | scarecrow 1 (PAT1-like) | rice actin | | PMON68887 |
| 113 | PHE0000128 | 218 | scarecrow 2 | rice actin | | — |
| 114 | PHE0000133 | 223 | G protein b subunit | rice actin | | PMON68860 |
| 115 | PHE0000152 | 242 | 14-3-3-like protein 2 | rice actin | | PMON77899 |
| 116 | PHE0000153 | 243 | 14-3-3-like protein D | rice actin | | PMON67817 |
| 117 | PHE0000154 | 244 | 14-3-3 protein 1 | rice actin | | PMON67818 |
| 118 | PHE0000155 | 245 | Rice FAP1-like protein | rice actin | | — |
| 119 | PHE0000156 | 246 | rice TAP42-like | rice actin | | — |
| 120 | PHE0000158 | 248 | BMH1 | rice actin | | PMON73169 |
| 121 | PHE0000159 | 250 | rice chloroplastic fructose-1,6- | rice actin | | PMON83640 |
| | | | bisphosphatase | | | |
| 122 | PHE0000160 | 251 | E. coli fructose-1,6- | rice actin | chloroplast | PMON75485 |
| | | | bisphosphatase | | | |
| 123 | PHE0000161 | 252 | Synechocystis fructose-1,6- | rice actin | chloroplast | PMON82231 |
| | | | bisphosphatase F-I | | | |
| 124 | PHE0000162 | 3383 | Synechocystis fructose-1,6- | Glutellin1 | | — |
| | | | bisphosphatase F-II | 3.1 kb | | |
| 124 | PHE0000162 | 253 | Synechocystis fructose-1,6- | rice actin | chloroplast | PMON75488 |
| | | | bisphosphatase F-II | | | |
| 125 | PHE0000164 | 255 | Yeast RPT5 | rice actin | | PMON73170 |
| 126 | PHE0000165 | 257 | Yeast RRP5 | rice actin | | PMON81210 |
| 127 | PHE0000166 | 258 | Rice CBP-like gene | rice actin | | — |
| 128 | PHE0000167 | 259 | rice BAB09754 | rice actin | | PMON80340 |
| 129 | PHE0000168 | 260 | LIB3061-001-H7_FLI | rice actin | | PMON68857 |
| 130 | PHE0000169 | 262 | maize p23 | rice actin | | — |
| 131 | PHE0000170 | 263 | maize cyclophilin | rice actin | | PMON81258 |
| 132 | PHE0000172 | 265 | yeast SIT1 | rice actin | | PMON81206 |
| 133 | PHE0000173 | 266 | yeast CNS1 | rice actin | | PMON73171 |
| 134 | PHE0000176 | 269 | RNAse S | rice actin | | PMON68388 |
| 135 | PHE0000177 | 270 | maize ecto-apyrase | rice actin | | PMON68881 |
| 136 | PHE0000178 | 271 | PHO5 | rice actin | | PMON73166 |
| 137 | PHE0000179 | 272 | high affinity phosphate | rice actin | | PMON69467 |
| | | | translocator | | | |
| 138 | PHE0000180 | 273 | high affinity phosphate | rice actin | | PMON83753 |
| | | | translocator | | | |
| 139 | PHE0000181 | 274 | Xylella citrate synthase | rice actin | | PMON76326 |
| 140 | PHE0000182 | 275 | E. coli citrate synthase | rice actin | | PMON74420 |
| 141 | PHE0000183 | 276 | rice citrate synthase | rice actin | | PMON80258 |
| 142 | PHE0000184 | 277/4429 | citrate synthase | rice actin | | PMON81278 |
| 143 | PHE0000185 | 278 | citrate synthase | rice actin | | PMON69468 |
| 144 | PHE0000186 | 279 | maize ferritin 2 | rice actin | | PMON69460 |
| 145 | PHE0000187 | 280 | maize ferritin 1 | rice actin | | PMON81261 |
| 146 | PHE0000188 | 281 | E. coli cytoplasmic ferritin | rice actin | | PMON73167 |
| 147 | PHE0000190 | 283 | corn LEA3 | rice actin | | — |
| 148 | PHE0000192 | 285 | soy HSF | rice actin | | PMON68394 |
| 149 | PHE0000193 | 286 | soy HSF | rice actin | | PMON68889 |
| 150 | PHE0000204 | 297 | deoxyhypusine synthase | rice actin | | — |
| 151 | PHE0000219 | 308 | thylakoid carbonic anhydrase, | rice actin | | PMON68865 |
| | | | cah3 | | | |
| 152 | PHE0000216 | 309 | thylakoid carbonic anhydrase, | rice actin | | PMON81823 |
| | | | ecaA | | | |
| 153 | PHE0000217 | 310 | Chlamydomonas reinhardtii | rice actin | | — |
| | | | envelope protein LIP-36G1 | | | |
| 154 | PHE0000218 | 311/4431 | psbO transit | rice actin | chloroplast | — |
| | | | peptide::Synechococcus sp. PCC | | lumen | |
| | | | 7942 ictB | | | |
| 155 | PHE0000220 | 313 | corn RNase PH | rice actin | | PMON74434 |
| 156 | PHE0000221 | 314 | SKI2 | rice actin | | — |
| 157 | PHE0000222 | 315 | SKI3 | rice actin | | PMON80320 |
| 158 | PHE0000223 | 316 | SKI4 | rice actin | | PMON69478 |
| 159 | PHE0000224 | 317 | SKI6 | rice actin | | PMON80278 |
| 160 | PHE0000225 | 318 | SKI7 | rice actin | | — |
| 161 | PHE0000226 | 319 | rice SKI7-like | rice actin | | — |
| 162 | PHE0000228 | 321 | Synechocystis cobA w cp transit | rice actin | chloroplast | — |
| | | | peptide | | | |
| 163 | PHE0000229 | 324 | Xylella tetrapyrrole methylase | rice actin | chloroplast | PMON77900 |
| | | | with transit peptide | | | |
| 164 | PHE0000230 | 325 | maize uroporphyrinogen III | rice actin | | — |
| | | | methyltransferase | | | |
| 165 | PHE0000231 | 326 | nucellin-like protein | rice actin | | PMON72498 |
| 166 | PHE0000232 | 327 | nucellin-like protein | rice actin | | PMON68895 |
| 167 | PHE0000233 | 328 | nucellin-like protein | rice actin | | PMON82671 |
| 168 | PHE0000234 | 329 | soy LEA protein | rice actin | | PMON73159 |
| 169 | PHE0000235 | 330 | dehydrin-like protein | rice actin | | PMON73161 |
| 170 | PHE0000237 | 332 | dehydrin 3 | rice actin | | PMON68891 |
| 171 | PHE0000238 | 333 | probable lipase | rice actin | | PMON69466 |
| 172 | PHE0000239 | 334 | yeast GRE1 | rice actin | | PMON72466 |
| 173 | PHE0000240 | 335 | yeast STF2 | rice actin | | PMON72468 |
| 174 | PHE0000241 | 336 | yeast SIP18 | rice actin | | — |
| 175 | PHE0000242 | 337 | yeast YBM6 | rice actin | | PMON72470 |
| 176 | PHE0000243 | 338 | yeast HSP12 | rice actin | | PMON72467 |
| 177 | PHE0000249 | 340 | corn allene oxide synthase | rice actin | | PMON74422 |
| 178 | PHE0000250 | 341 | corn COI1-like | rice actin | | PMON82194 |
| 179 | PHE0000251 | 342 | corn TIR1-like | rice actin | | — |
| 180 | PHE0000252 | 343 | corn COI1-like | rice actin | | PMON74407 |
| 181 | PHE0000253 | 344 | COI1-like | rice actin | | — |
| 182 | PHE0000254 | 345 | F-box protein | rice actin | | PMON73172 |
| 183 | PHE0000255 | 346 | F-box protein | rice actin | | PMON72459 |
| 184 | PHE0000256 | 347 | corn 1-aminocyclopropane-1- | rice actin | | PMON75302 |
| | | | carboxylate oxidase | | | |
| 185 | PHE0000257 | 348 | rice 1-aminocyclopropane-1 | rice actin | | PMON80260 |
| | | | carboxylate synthase | | | |
| 186 | PHE0000260 | 349 | S52650 - Synechocystis desB | rice actin | chloroplast | PMON75487 |
| 187 | PHE0000261 | 350 | yeast glutamate decarboxylase | rice actin | | PMON80276 |
| 188 | PHE0000262 | 352 | cytochrome P450-like protein | rice actin | | PMON68892 |
| 189 | PHE0000263 | 353 | cytochrome P450 | rice actin | | PMON74412 |
| 190 | PHE0000264 | 354 | cytochrome P450-like | rice actin | | PMON68866 |
| 191 | PHE0000265 | 355 | CYP90 protein | rice actin | | PMON69469 |
| 192 | PHE0000266 | 356 | cytochrome P450 DWARF3 | rice actin | | PMON69470 |
| 193 | PHE0000267 | 357 | cytochrome P450 | rice actin | | PMON68867 |
| 194 | PHE0000268 | 358 | rice receptor protein kinase | rice actin | | — |
| 195 | PHE0000269 | 359 | soy E2F-like | rice actin | | — |
| 196 | PHE0000270 | 360/4432 | nuclear matrix constituent protein | rice actin | | PMON80316 |
| 197 | PHE0000271 | 361/4433 | OsE2F1 | rice actin | | — |
| 198 | PHE0000272 | 362 | corn GCR1 | rice actin | | — |
| 199 | PHE0000273 | 363 | soy mlo-like | rice actin | | PMON74423 |
| 200 | PHE0000274 | 364 | soy mlo-like | rice actin | | — |
| 201 | PHE0000275 | 365 | rice G alpha 1 | rice actin | | PMON80259 |
| 202 | PHE0000276 | 366 | soy G-gamma subunit | rice actin | | PMON68868 |
| 203 | PHE0000277 | 368 | wheat G28-like | rice actin | | PMON68890 |
| 204 | PHE0000279 | 369 | sorghum proline permease | rice actin | | PMON68896 |
| 205 | PHE0000280 | 370 | rice AA transporter | rice actin | | PMON72451 |
| 206 | PHE0000282 | 372 | SET-domain protein-like | rice actin | | — |
| 207 | PHE0000283 | 373 | scarecrow 6 | rice actin | | PMON69472 |
| 208 | PHE0000284 | 374 | menage a trois-like | rice actin | | PMON72453 |
| 209 | PHE0000286 | 376 | oryzacystatin | rice actin | | PMON72454 |
| 210 | PHE0000287 | 377 | Similar to cysteine proteinase | rice actin | | PMON68898 |
| | | | inhibitor | | | |
| 211 | PHE0000288 | 378 | cysteine proteinase inhibitor | rice actin | | — |
| 212 | PHE0000289 | 379 | Zm-GRF1 (GA responsive factor) | rice actin | | — |
| 213 | PHE0000290 | 380 | ZmSE001-like | rice actin | | — |
| 214 | PHE0000291 | 381 | deoxyhypusine synthase | rice actin | | PMON72455 |
| 215 | PHE0000293 | 382/4368 | gibberellin response modulator | rice actin | | PMON75972 |
| 216 | PHE0000294 | 383 | scarecrow-like protein | rice actin | | PMON68897 |
| 217 | PHE0000295 | 384 | ubiquitin-conjugating enzyme- | rice actin | | PMON68894 |
| | | | like protein | | | |
| 218 | PHE0000296 | 385 | unknown protein recognized by | rice actin | | PMON68893 |
| | | | PF01169 | | | |
| 219 | PHE0000297 | 386 | 26S protease regulatory subunit | rice actin | | PMON68899 |
| | | | 6A homolog | | | |
| 220 | PHE0000298 | 387 | rice p23 co-chaperone | rice actin | | PMON68874 |
| 221 | PHE0000299 | 388 | corn p23 co-chaperone | rice actin | | PMON68875 |
| 222 | PHE0000300 | 389 | rice p23 co-chaperone | rice actin | | PMON68876 |
| 223 | PHE0000301 | 390 | corn p23 co-chaperone | rice actin | | PMON68877 |
| 224 | PHE0000302 | 391 | putative purple acid phosphatase | rice actin | | PMON68878 |
| | | | precursor | | | |
| 225 | PHE0000303 | 392 | acid phosphatase type 5 | rice actin | | PMON68879 |
| 226 | PHE0000304 | 393 | aleurone ribonuclease | rice actin | | PMON68873 |
| 227 | PHE0000305 | 394 | putative ribonuclease | rice actin | | PMON68880 |
| 228 | PHE0000306 | 395 | S-like RNase | rice actin | | PMON68882 |
| 229 | PHE0000307 | 396 | ribonuclease | rice actin | | PMON68883 |
| 230 | PHE0000308 | 397 | helix-loop-helix protein (PIF3- | rice actin | | PMON68884 |
| | | | like) | | | |
| 231 | PHE0000309 | 398 | SKI4-like protein | rice actin | | PMON68885 |
| 232 | PHE0000310 | 399 | putative 3 exoribonuclease | rice actin | | PMON68377 |
| 233 | PHE0000311 | 400 | GF14-c protein | rice actin | | PMON72458 |
| 234 | PHE0000312 | 401 | 14-3-3-like protein | rice actin | | PMON72456 |
| 235 | PHE0000313 | 402 | rice eIF-(iso)4F | rice actin | | PMON68378 |
| 236 | PHE0000314 | 403 | rice eIF-4F | rice actin | | PMON68379 |
| 237 | PHE0000315 | 404 | sorghum eIF-(iso)4F | rice actin | | PMON68381 |
| 238 | PHE0000316 | 405 | sorghum eIF-4F | rice actin | | PMON68382 |
| 239 | PHE0000317 | 406 | rice FIP37-like | rice actin | | PMON68380 |
| 240 | PHE0000318 | 407 | scarecrow 17 | rice actin | | PMON81878 |
| 241 | PHE0000322 | 411 | maize catalase-1 | rice actin | | PMON74403 |
| 242 | PHE0000323 | 412 | maize catalase-3 | rice actin | | PMON68400 |
| 243 | PHE0000324 | 413 | ascorbate peroxidase | rice actin | | PMON73162 |
| 244 | PHE0000325 | 414 | corn GDI | rice actin | | PMON68384 |
| 245 | PHE0000326 | 415 | soy GDI | rice actin | | PMON72463 |
| 246 | PHE0000327 | 416 | corn rho GDI | rice actin | | PMON69481 |
| 247 | PHE0000328 | 417 | basic blue copper protein | rice actin | | PMON74416 |
| 248 | PHE0000329 | 418 | plantacyanin | rice actin | | PMON80945 |
| 249 | PHE0000330 | 419 | basic blue copper protein | rice actin | | PMON73164 |
| 250 | PHE0000331 | 420 | Similar to blue copper protein | rice actin | | — |
| | | | precursor | | | |
| 251 | PHE0000332 | 421 | lamin | rice actin | | PMON68385 |
| 252 | PHE0000333 | 422 | fC-zmfl700551169a-allyl alcohol | rice actin | | PMON75470 |
| | | | dehydrogenase | | | |
| 253 | PHE0000334 | 423 | allyl alcohol dehydrogenase | rice actin | | PMON68395 |
| 254 | PHE0000335 | 424 | allyl alcohol dehydrogenase | rice actin | | PMON74413 |
| 255 | PHE0000336 | 425 | qui oxidoreductase | rice actin | | PMON74414 |
| 256 | PHE0000337 | 426 | E. nidulans cysA - AF029885 | rice actin | | — |
| 257 | PHE0000338 | 427 | BAA18167 - Synechocystis cysE | rice actin | | PMON68628 |
| 258 | PHE0000339 | 428 | Synechocystis thiol-specific | rice actin | | PMON68627 |
| | | | antioxidant protein - BAA10136 | | | |
| 258 | PHE0000339 | 429 | Synechocystis thiol-specific | rice actin | chloroplast | PMON75490 |
| | | | antioxidant protein - BAA10136 | | | |
| 259 | PHE0000340 | 430 | yeast TSA2 - NP_010741 | rice actin | | PMON68629 |
| 260 | PHE0000341 | 431 | yeast mTPx - Z35825 | rice actin | | PMON68397 |
| 261 | PHE0000343 | 433 | yeast TPx III - NP_013210 | rice actin | | PMON80506 |
| 262 | PHE0000345 | 435 | soy putative 2-cys peroxiredoxin | rice actin | | PMON74411 |
| 263 | PHE0000346 | 436 | soy peroxiredoxin | rice actin | | PMON73165 |
| 264 | PHE0000347 | 437 | heat shock protein 26, plastid- | rice actin | | PMON68386 |
| | | | localized | | | |
| 265 | PHE0000349 | 439 | heat shock protein | rice actin | | PMON68389 |
| 266 | PHE0000350 | 440 | low molecular weight heat shock | rice actin | | PMON74410 |
| | | | protein | | | |
| 267 | PHE0000351 | 441 | 18 kDa heat shock protein | rice actin | | — |
| 268 | PHE0000352 | 442 | heat shock protein 16.9 | rice actin | | PMON74409 |
| 269 | PHE0000353 | 443 | HSP21-like protein | rice actin | | PMON73160 |
| 270 | PHE0000354 | 444 | Opt1p - NP_012323 | rice actin | | PMON81879 |
| 271 | PHE0000355 | 445 | SVCT2-like permease | rice actin | | PMON83797 |
| 272 | PHE0000356 | 446 | SVCT2-like permease | rice actin | | PMON72464 |
| 273 | PHE0000357 | 447 | maize tubby-like | rice actin | | PMON69474 |
| 274 | PHE0000358 | 449 | maize tubby-like | rice actin | | PMON69475 |
| 275 | PHE0000359 | 450 | soy HMG CoA synthase | rice actin | | PMON69476 |
| 276 | PHE0000360 | 451 | yeast HMGS - X96617 | rice actin | | PMON81886 |
| 277 | PHE0000361 | 452 | PAT1-like scarecrow 9 | rice actin | | PMON78900 |
| 278 | PHE0000362 | 453 | CDC28-related protein kinase | rice actin | | PMON81840 |
| 279 | PHE0000385 | 474 | H+ transporting ATPase | rice actin | | PMON75498 |
| 280 | PHE0000386 | 475 | cation-transporting ATPase | rice actin | | PMON67834 |
| 281 | PHE0000387 | 476 | yeast DRS2 (ALA1-like) - | rice actin | | — |
| | | | L01795 | | | |
| 282 | PHE0000388 | 477 | S. pombe ALA1-like-CAA21897 | rice actin | | — |
| 283 | PHE0000389 | 478 | rice ALA1-like 1 - BAA89544 | rice actin | | PMON80290 |
| 284 | PHE0000390 | 479/4340 | rice chloroplastic sedoheptulose- | rice actin | | PMON67836 |
| | | | 1,7-bisphosphatase- | | | |
| 285 | PHE0000391 | 480/4434 | rice cytosolic fructose-1,6- | rice actin | | PMON67835 |
| | | | bisphosphatase | | | |
| 286 | PHE0000392 | 481 | Wheat sedoheptulose-1,7- | rice actin | | PMON76335 |
| | | | bisphosphatase | | | |
| 287 | PHE0000394 | 483 | sedoheptulose-1,7-bisphosphatase | rice actin | | — |
| 288 | PHE0000395 | 484 | soy phantastica (rough sheath 2- | rice actin | | PMON67840 |
| | | | like) | | | |
| 289 | PHE0000396 | 485 | soy phantastica 2 (rough sheath 2- | rice actin | | PMON67838 |
| | | | like) | | | |
| 290 | PHE0000397 | 486 | maize rough sheath 1 | rice actin | | PMON67839 |
| 291 | PHE0000398 | 487 | soy lg3-like 1 | rice actin | | PMON72488 |
| 292 | PHE0000399 | 488 | soy rough sheath1-like 1 | rice actin | | PMON72485 |
| 293 | PHE0000400 | 489 | soy G559-like | rice actin | | PMON72486 |
| 294 | PHE0000401 | 490 | soy G1635-like 1 | rice actin | | PMON67837 |
| 295 | PHE0000402 | 491 | rice amino acid transporter-like | rice actin | | PMON67833 |
| | | | protein | | | |
| 296 | PHE0000403 | 492/4341 | corn amino acid permease | rice actin | | PMON67831 |
| 297 | PHE0000404 | 493 | rice proline transport protein | rice actin | | PMON67832 |
| 298 | PHE0000412 | 501 | corn monosaccharide transporter 1 | rice actin | | PMON67843 |
| 299 | PHE0000413 | 502 | soy monosaccharide transporter 3 | rice actin | | PMON67844 |
| 300 | PHE0000414 | 503 | corn monosaccharide transporter 3 | rice actin | | PMON67845 |
| 301 | PHE0000415 | 504 | soy monosaccharide transporter 1 | rice actin | | PMON67846 |
| 302 | PHE0000416 | 505 | corn monosaccharide transporter 6 | rice actin | | PMON67847 |
| 303 | PHE0000418 | 507 | corn monosaccharide transporter 4 | rice actin | | PMON69497 |
| 304 | PHE0000419 | 508 | soy monosaccharide transporter 2 | rice actin | | PMON67848 |
| 305 | PHE0000420 | 509 | soy sucrose transporter | rice actin | | PMON74415 |
| 306 | PHE0000421 | 510 | corn sucrose transporter 2 | rice actin | | PMON83760 |
| 307 | PHE0000422 | 511 | corn monosaccharide transporter 8 | rice actin | | PMON79433 |
| 308 | PHE0000423 | 512 | corn monosaccharide transporter 7 | rice actin | | PMON72497 |
| 309 | PHE0000425 | 514 | soy isoflavone synthase | rice actin | | PMON72495 |
| 310 | PHE0000426 | 515 | soy ttg1-like 2 | rice actin | | PMON74408 |
| 311 | PHE0000427 | 516 | GATE5 - corn SPA1-like 1 | rice actin | | — |
| 312 | PHE0000428 | 517 | corn PIF3-like | rice actin | | PMON74417 |
| 313 | PHE0000429 | 518 | soy Athb-2-like 1 | rice actin | | PMON74418 |
| 314 | PHE0000430 | 519 | corn SUB1-like 1 | rice actin | | — |
| 315 | PHE0000431 | 520/4435 | soy GH3 protein | rice actin | | PMON81262 |
| 316 | PHE0000432 | 521 | corn 12-oxophytodienoate | rice actin | | PMON79441 |
| | | | reductase 1 | | | |
| 317 | PHE0000433 | 522 | corn 12-oxo-phytodienoate | rice actin | | PMON74424 |
| | | | reductase-like 3 | | | |
| 318 | PHE0000434 | 523 | corn 12-oxophytodienoate | rice actin | | PMON74419 |
| | | | reductase-like 4 | | | |
| 319 | PHE0000435 | 524 | corn hydroperoxide lyase | rice actin | | PMON75499 |
| 320 | PHE0000436 | 525 | rice cns1-like | rice actin | | PMON79442 |
| 321 | PHE0000437 | 526 | corn HCH1-like 1 | rice actin | | PMON68630 |
| 322 | PHE0000438 | 527 | corn HOP-like 1 | rice actin | | PMON74433 |
| 323 | PHE0000439 | 528 | corn HOP-like 2 | rice actin | | PMON74425 |
| 324 | PHE0000440 | 529 | rice CHIP-like 1 | rice actin | | PMON72473 |
| 325 | PHE0000441 | 530 | corn CHIP-like 2 | rice actin | | PMON72474 |
| 326 | PHE0000451 | 540 | wheat SVP-like 1 | rice actin | | PMON72475 |
| 327 | PHE0000452 | 541 | corn SVP-like 3 | rice actin | | PMON72476 |
| 328 | PHE0000453 | 542 | corn SVP-like 5 | rice actin | | — |
| 329 | PHE0000454 | 543 | fC-zmhuLIB3062-044-Q1-K1-B8 | rice actin | | PMON72477 |
| 330 | PHE0000455 | 544 | corn E4/E8 binding protein-like | rice actin | | — |
| 331 | PHE0000469 | 558 | yeast YKL091c - Z28091 | rice actin | | PMON68636 |
| 332 | PHE0000470 | 559 | corn Ssh1-like protein 1 | rice actin | | PMON79435 |
| 333 | PHE0000471 | 560 | corn Ssh1-like protein 3 | rice actin | | PMON73772 |
| 334 | PHE0000472 | 561 | corn Ssh1-like protein 4 | rice actin | | PMON79436 |
| 335 | PHE0000473 | 562 | soy Ssh1-like protein 2 [ssh2] | rice actin | | PMON75471 |
| 336 | PHE0000484 | 573 | soy JMT-like protien 1 | rice actin | | PMON81287 |
| 337 | PHE0000485 | 574 | corn JMT-like protein 1 | rice actin | | PMON69498 |
| 338 | PHE0000486 | 575 | corn JMT-like protein 2 | rice actin | | PMON69496 |
| 339 | PHE0000017 | 27 | corn AAA-ATPase 1 | rice actin | | PMON68850 |
| 339 | PHE0000017 | 686 | corn AAA-ATPase 1 | rice actin | | PMON72479 |

### Example 2

### Constructs for Soybean Transformation

Constructs for use in transformation of soybean may be prepared by restriction enzyme based cloning into a common expression vector. Elements of an exemplary common expression vector are shown in Table 7 below.

TABLE 7
Elements of pMON74532

| Function | Element | Reference |
|---|---|---|
| Agro transformation | B-ARGtu.right border | Depicker, A. et al (1982) |
| | | Mol Appl Genet 1: 561-573 |
| Antibiotic resistance | CR-Ec.aadA-SPC/STR | |
| Repressor of primers from the ColE1 | CR-Ec.rop | |
| plasmid | | |
| Origin of replication | OR-Ec.oriV-RK2 | |
| Agro transformation | B-ARGtu.left border | Barker, R. F. et al (1983) |
| | | Plant Mol Biol 2: 335-350 |
| Plant selectable marker expression | Promoter with intron and | McDowell et al. (1996) |
| cassette | 5′UTR of arabidopsis act 7 | Plant Physiol. 111: 699-711. |
| | gene (AtAct7) | |
| | 5′ UTR of arabidopsis act 7 | |
| | gene | |
| | Intron in 5′UTR of AtAct7 | |
| | Transit peptide region of | Klee, H. J. et al (1987) |
| | Arabidopsis EPSPS | MGG 210: 437-442 |
| | Synthetic CP4 coding region | U.S. Pat. No. 6,248,876 |
| | with dicot preferred codon | |
| | usage | |
| | A 3′ UTR of the nopaline | U.S. Pat. No. 5,858,742 |
| | synthase gene of | |
| | Agrobacterium tumefaciens | |
| | Ti plasmid | |
| Plant gene of interest expression | Promoter for 35S RNA from | U.S. Pat. No. 5,322,938 |
| cassette | CaMV containing a | |
| | duplication of the −90 to −350 | |
| | region (e35S) | |
| | Gene of interest insertion site | |
| | Cotton E6 3′ end | GenBank accession |
| | | U30508 |

Vectors similar to that described above may be constructed for use in Agrobacterium mediated soybean transformation systems where the enhanced 35S promoter in the plant expression cassette portion is replaced with other desirable promoters including, but not limited to a napin promoter and an Arabidopsis SSU promoter. Protein coding segments are amplified by PCR prior to insertion into vectors such as described above. Primers for PCR amplification can be designed at or near the start and stop codons of the coding sequence, in order to eliminate most of the 5′ and 3′ untranslated regions.

Exemplary sense constructs for transformation of soybean to produce plants having enhanced phenotypes are provided in Table 8 below. Column headings in Table 8 refer to the following information:

“SEQ ID NO” refers to a particular nucleic acid sequence in the Sequence Listing which defines a polynucleotide used in a recombinant polynucleotide of this invention.

“PHE ID” refers to an arbitrary number used to identify a particular recombinant polynucleotide corresponding to the translated protein encoded by the polynucleotide.

“NOM ID” refers to a particular construct comprising a polynucleotide of this invention.

“GENE NAME” refers to a common name for the recombinant polynucleotide.

“PROMOTER” provides the name of the promoter region driving expression of the polynucleotide

“pMON” refers to an arbitrary number used to designate a particular recombinant DNA construct. Constructs are prophetic where no pMON is provided.

“Gene effect contributing to increased yield” describes the effect of the recombinant polynucleotide on the plant in providing yield improvement.

TABLE 8
Soybean Transformation Constructs

| SEQ | | | | | | |
|---|---|---|---|---|---|---|
| ID | | Nom | | | | Gene effect contributing |
| NO | Phe ID | ID | Gene Name | Promoter | pMON | to increased yield |
| 8 | PHE0000012 | 4249 | corn aquaporin RS81 | e35S | pMON 83080 | Increased root mass |
| 34 | PHE0000040 | 3968 | corn hemoglobin 1 | e35S | pMON 83103 | Cold tolerance |
| 53 | PHE0000057 | 3962 | corn mt NDK - | e35S | pMON 83055 | Abiotic stress tolerance |
| | | | LIB189022Q1E1E9 | | | |
| 62 | PHE0000067 | 4248 | yeast eIF-5A | e35S | pMON83076 | Nitrogen use efficiency |
| 123 | PHE0000161 | 3578 | Synechocystis fructose- | e35S | pMON 81321 | Increased sucrose |
| | | | 1,6-bisphosphatase F-I | | | production/transport |
| 204 | PHE0000279 | 4246 | sorghum proline permease | e35S | pMON 83093 | Nitrogen use efficiency |
| 234 | PHE0000312 | 4247 | 14-3-3-like protein | e35S | pMON83075 | Nitrogen use efficiency |
| 236 | PHE0000314 | 4245 | rice eIF-4F | e35S | pMON83074 | Nitrogen use efficiency |
| 253 | PHE0000334 | 4268 | allyl alcohol | e35S | pMON 84409 | Heat tolerance |
| | | | dehydrogenase | | | |

### Example 3

### Plant Transformation

Maize Transformation

LH59 plants are grown in the greenhouse and ears and ears harvested when the embryos are 1.5 to 2.0 mm in length. Ears were surface sterilized by spraying or soaking the ears in 80% ethanol, followed by air drying. Immature embryos were isolated from individual kernels on surface sterilized ears. Prior to inoculation of maize cells, Agrobacterium cells are grown overnight at room temperature. Immature maize embryos are inoculated with Agrobacterium shortly after excision, and incubated at room temperature with Agrobacterium for 5-20 minutes. Immature embryos are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing 100 mg/L paromomycin and subcultured at about two week intervals. Transformants are recovered 6 to 8 weeks after initiation of selection.

For Agrobacterium mediated transformation of maize callus, immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media, and paromomycin resistant callus identified.

For transformation by microprojectile bombardment, immature maize embryos are isolated and cultured 3-4 days prior to bombardment. Prior to microprojectile bombardment, a suspension of gold particles is prepared onto which the desired DNA is precipitated. DNA is introduced into maize cells as described in U.S. Pat. No. 5,015,580 using the electric discharge particle acceleration gene delivery device. For microprojectile bombardment of LH59 pre-cultured immature embryos, 35% to 45% of maximum voltage is preferably used. Following microprojectile bombardment, tissue is cultured in the dark at 27° C.

Fertile transgenic plants are produced from transformed maize cells by transfer of. transformed callus to appropriate regeneration media to initiate shoot development. Plantlets are transferred to soil when they are about 3 inches tall and have roots (about four to 6 weeks after transfer to medium). Plants are maintained for two weeks in a growth chamber at 26° C., followed by two weeks on a mist bench in a greenhouse before transplanting to 5 gallon pots for greenhouse growth. Plants are grown in the greenhouse to maturity and reciprocal pollinations made with the inbred LH59. Seed is collected from plants and used for further breeding activities.

Transformation methods and materials for making transgenic plants of this invention, e.g. various media and recipient target cells, transformation of immature embryos and subsequent regeneration of fertile transgenic plants are disclosed in U.S. Pat. Nos. 6,194,636 and 6,232,526 and U.S. patent application Ser. No. 09/757,089, which are incorporated herein by reference.

Soybean Transformation

For Agrobacterium mediated transformation, soybean seeds are germinated overnight and the meristem explants excised. The meristems and the explants are placed in a wounding vessel. Soybean explants and induced Agrobacterium cells from a strain containing plasmid DNA with the gene of interest cassette and a plant selectable marker cassette are mixed no later than 14 hours from the time of initiation of seed germination and wounded using sonication. Following wounding, explants are placed in co-culture for 2-5 days at which point they are transferred to selection media for 6-8 weeks to allow selection and growth of transgenic shoots. Phenotype positive shoots are harvested approximately 6-8 weeks post bombardment and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil.

Descriptions of media useful for transformation and regeneration of soybean and a method employing microprojectile bombardment are described in U.S. Pat. No. 5,914,451, which is incorporated herein by reference.

### Example 4

### Identification of Homologs

A BLAST searchable “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism. Nucleotide sequences of genes provided herein are identified by SEQ ID NO in Table 1. SEQ ID NOs of amino acid sequences and organism name for polypeptides encoded by the polynucleotides provided herein are shown in Table 2.

The All Protein Database was queried using polypeptide sequences provided herein as SEQ ID NO: 340 through SEQ ID NO:678 using “blastp” with E-value cutoff of 1e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 340 through SEQ ID NO:678 using “blastp” with E-value cutoff of 1e-4. Up to 1000 top hits were kept. A BLAST searchable database was constructed based on these hits, and is referred to as “SubDB”. SubDB was queried with each sequence in the Hit List using “blastp” with E-value cutoff of 1e-8. The hit with the best E-value was compared with the Core List from the corresponding organism. The hit is deemed a likely ortholog if it belongs to the Core List, otherwise it is deemed not a likely oththolog and there is no further search of sequences in the Hit List for the same organism. Likely orthologs from a large number of distinct organisms were identified and are reported by amino acid sequences of SEQ ID NO:679 to SEQ ID NO: 24149. These orthologs are reported in Table 2 as homologs to the 339 polypeptides provided herein. Table 3 provides the SEQ ID NO and the name of the organism in which it was identified for each homolog gene.

All publications and patent applications cited herein are incorporated by reference in their entirely to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. Transgenic plant seed, wherein the genome of said seed comprises a recombinant polynucleotide encoding a polypeptide selected from the group consisting of S-adenosylmethionine decarboxylase and deoxyhypusine synthase, and wherein plants grown from said seed exhibit enhanced yield.

2-4. (canceled)

5. Transgenic plant seed of claim 1, wherein said polypeptide has an amino acid sequence that is at least 70% identical to an amino acid sequence selected from the group consisting of SEQ ID NO:456, 457, 458 459, 460 and 452, wherein identity is determined by calculating the percentage of identical and conservatively substituted amino acids in the homolog over the length of the SEQ ID.

6. Transgenic plant seed of claim 1, wherein said homolog has an amino acid sequence selected from the group consisting of SEQ ID NO:679 through SEQ ID NO:24149.

7. Transgenic plant seed of claim 1, wherein said polypeptide has an amino acid sequence selected from the group consisting of SEQ ID NO: 456, 457, 458 459, 460 and 452.

8. Transgenic plant seed of claim 1, wherein said seed is from a maize plant or a soybean plant.

9. A method of producing a plant having an enhanced phenotype, wherein said method comprises transforming plant cells with a recombinant polynucleotide comprising a promoter functional in a plant cell operably joined to encoding sequence for a polypeptide selected from the group consisting of S-adenosylmethionine decarboxylase and deoxyhypusine synthase, regenerating plants from said cells, and screening said plants to identify a plant having an enhanced phenotype.

10. A method of claim 9, wherein said enhanced phenotype is increased yield.

11-12. (canceled)

13. A method of claim 9, wherein said homolog has an amino acid sequence that is at least 70% identical to an amino acid sequence selected from the group consisting of SEQ ID NO: 456, 457, 458 459, 460 and 452.

14. A method of claim 9, wherein said homolog has an amino acid sequence selected from the group consisting of SEQ ID NO:679 through SEQ ID NO:24149.

15. A method of claim 9, wherein said polypeptide has an amino acid sequence selected from the group consisting of SEQ ID NO: 456, 457, 458 459, 460 and 452.

16. A method of claim 9, wherein said plant is a maize plant or a soybean plant.

17. A recombinant polynucleotide comprising a promoter functional in a plant cell operably joined to encoding sequence for a polypeptide having an amino acid selected from the group consisting of SEQ ID NO: 456, 457, 458 459, 460 and 452 and homologs thereof.

18. A recombinant polynucleotide of claim 17, wherein said homolog has an amino acid sequence that is at least 70% identical to an amino acid sequence selected from the group consisting of SEQ ID NO: 456, 457, 458 459, 460 and 452.

19. A recombinant polynucleotide of claim 17, wherein said homolog has an amino acid sequence selected from the group consisting of SEQ ID NO:679 through SEQ ID NO:24149.

20. A recombinant polynucleotide of claim 17, wherein said polypeptide has an amino acid sequence selected from the group consisting of SEQ ID NO: 456, 457, 458 459, 460 and 452.

21. A recombinant polynucleotide of claim 17, wherein said promoter is selected from the group consisting of a rice actin promoter, a glutelin 1 promoter and a PPDK promoter.
